(19) United States
(12) Patent Application Publication
(10) Pub. No.: US 2010/0055700 A1
(43) Pub. Date: Mar. 4, 2010

(54) ROLE OF IL-12, IL-23 AND IL-17 RECEPTORS IN INFLAMMATORY BOWEL DISEASE

(75) Inventors: Stephan R. Targan, Santa Monica, CA (US); Jerome I. Rotter, Los Angeles, CA (US); Kent D. Taylor, Ventura, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/528,668

(22) PCT Filed: Feb. 28, 2008
(86) PCT No.: PCT/US08/55236

§ 371 Date: Aug. 26, 2009

Related U.S. Application Data
(60) Provisional application No. 60/892,165, filed on Feb. 28, 2007.

(51) Int. Cl. C12Q 1/68

(52) U.S. Cl. 435/6

(57) ABSTRACT

This invention provides methods of diagnosing or predicting susceptibility or protection against Inflammatory Bowel Disease in an individual by determining the presence or absence of genetic variants in the genes for IL-12, IL-23, and/or IL-17 receptors. In one embodiment, a method of the invention is practiced by determining the presence or absence of risk and/or protective haplotypes of IL-12, IL-23, and/or IL-17 receptors.

### FIELD OF THE INVENTION

The invention relates generally to the fields of inflammation and autoimmunity and autoimmune disease and, more specifically, to genetic methods for diagnosing inflammatory bowel disease, Crohn's disease, and other autoimmune diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments provide methods of diagnosing susceptibility to Crohn's Disease in an individual, comprising determining the presence or absence of at least one risk haplotype at the IL23R locus selected from the group consisting of IL23R Block 2 H1 and IL23R Block 3 H1, where the presence of at least one risk haplotype at the IL23R locus is diagnostic of susceptibility to Crohn's Disease. In another embodiment, the individual may be a child and/or non-Jewish. In another embodiment, the IL23R Block 2 H1 further comprises one or more variant alleles selected from the group consisting of SEQ. ID. NO.: 9 and SEQ. ID. NO.: 10. In another embodiment, the IL23R Block 3 H1 further comprises one or more variant alleles selected from the group consisting of SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, and SEQ. ID. NO.: 18. In another embodiment, the presence of two of the risk haplotypes at the IL23R locus presents a greater susceptibility than the presence of one or none of the risk haplotypes at the IL23R locus, and the presence of one of the risk haplotypes at the IL23R locus presents a greater susceptibility than the presence of none of the risk haplotypes at the IL23R locus but less than the presence of the two risk haplotypes at the IL23R locus.

Other embodiments provide methods of diagnosing susceptibility to Crohn's Disease in an individual, comprising determining the presence or absence of one or more risk haplotypes at the IL23R locus, and determining the presence or absence of one or more risk haplotypes at the IL17A locus, where the presence of at least one risk haplotype at the IL23R locus and at least one risk haplotype at the IL17A locus is diagnostic of susceptibility of Crohn's Disease. In other embodiments, one of the one or more risk haplotypes at the IL93R locus may be IL23R Block 2 H1, and/or IL23R Block 3 H1. In another embodiment, one of the one or more risk haplotypes at the IL17A locus is IL17A H2. The IL17A H2 may further comprise one or more variant alleles selected from the group consisting of SEQ. ID. NO.: 19, SEQ. ID. NO.: 20, SEQ. ID. NO.: 21, SEQ. ID. NO.: 22, and SEQ. ID. NO.: 23.

Various embodiments provide methods of diagnosing susceptibility to Crohn's Disease in an individual, comprising determining the presence or absence of at least one risk haplotype at the IL23R locus, and determining the presence or absence of at least one risk haplotype at the IL17RA locus, where the presence of at least one risk haplotype at the IL23R locus and at least one risk haplotype at the IL17RA locus is diagnostic of susceptibility of Crohn's Disease. In other embodiments, one of the one or more risk haplotypes at the IL23R locus is IL23R Block 2 H1 and/or IL23R Block 3 H1. In other embodiments, one of the one or more risk haplotypes at the IL17RA locus is IL17RA Block 2 H4. The IL17RA Block 2 H4 may further comprise one or more variant alleles selected from the group consisting of SEQ. ID. NO.: 26, SEQ. ID. NO.: 27, SEQ. ID. NO.: 28, SEQ. ID. NO.: 29, SEQ. ID. NO.: 30, SEQ. ID. NO.: 31, and SEQ. ID. NO.: 32.

Other embodiments provide methods of determining a low probability relative to a healthy individual of developing Crohn's Disease in an individual, the method comprising determining the presence or absence of at least one protective haplotype at the IL23R locus selected from the group consisting of IL23R Block 3 H2 and IL23R Block 3 H6, where the presence of one or more of the protective haplotypes at the IL23R locus is diagnostic of the low probability relative to the healthy individual of developing Crohn's Disease. In other embodiments, the IL23R Block 3 H2 further comprises one or more variant alleles selected from the group consisting of SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, and SEQ. ID. NO.: 18. In other embodiments, the IL23R Block 3 H6 further comprise one or more variant alleles selected from the group consisting of SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, and SEQ. ID. NO.: 18.

The invention also provides embodiments of methods of diagnosing susceptibility to Crohn's Disease in an individual, comprising determining the presence or absence of one or more risk haplotypes at the IL17A locus in the individual, where the presence of one or more of the risk haplotypes is diagnostic of susceptibility to Crohn's Disease. One of the one or more risk haplotypes at the IL17A locus may be IL17A H2. In other embodiments, the individual is non-Jewish. In other embodiments, one of the one or more risk haplotypes at the IL17A locus may be IL17A H4. In other embodiments, the individual is Jewish.

Various embodiments provide methods of diagnosing susceptibility to inflammatory bowel disease in an individual, comprising determining the presence or absence of one or more risk haplotypes at the IL17RA locus in the individual, where the presence of one or more of said risk haplotypes is diagnostic of susceptibility to inflammatory bowel disease. One of the one or more risk haplotypes at the IL17RA locus may be IL17RA Block 2 H4. The inflammatory bowel disease may also comprise Crohn's Disease and/or ulcerative colitis.

Other embodiments provide methods of determining a low probability relative to a healthy individual of developing inflammatory bowel disease in an individual, the method comprising determining the presence or absence of one or more protective haplotypes at the IL17RA locus in the individual, where the presence of one or more of said protective haplotypes is diagnostic of the low probability relative to the healthy individual of developing inflammatory bowel disease. One of the one or more protective haplotypes at the IL17RA locus may be IL17RA Block 1 H3. The inflammatory bowel disease may also comprise Crohn's Disease and/or ulcerative colitis.

Various embodiments also provide methods of determining a low probability relative to a healthy individual of developing Crohn's Disease subtype, comprising determining the presence or absence of a IL12B(p40) H1 haplotype, where the presence of the IL12B(p40) H1 haplotype is diagnostic of a low probability relative to a healthy individual of developing Crohn's Disease. The IL12B(p40) H1 haplotype may also further comprise one or more variant alleles selected from the group consisting of SEQ. ID. NO.: 33, SEQ. ID. NO.: 34, SEQ. ID. NO.: 35, and SEQ. ID. NO.: 36.

Embodiments provide for methods of diagnosing a low probability relative to a healthy individual of developing Crohn's Disease, comprising determining the presence or absence of a IL12B(p40) H3 haplotype, and determining the presence or absence of Cbir1 antibody expression relative to an individual diagnosed with Crohn's Disease, where the presence of IL12B(p40) H3 haplotype and the absence of Cbir1 antibody expression relative to an individual diagnosed with Crohn's Disease is diagnostic of a low probability relative to a healthy individual of developing Crohn's Disease. The IL12B(p40) H3 haplotype may further comprise one or more variant alleles selected from the group consisting of SEQ. ID. NO.: 33, SEQ. ID. NO.: 34, SEQ. ID. NO.: 35, and SEQ. ID. NO.: 36.

Other embodiments provide methods of treating Crohn's Disease, comprising determining the presence or absence in the individual of one or more risk haplotypes selected from the group consisting of IL23R Block 2 H1, IL23R Block 3 H1, IL17A H2, and IL17RA Block 2 H4, and administering a therapeutically effective amount of treatment to the individual if the one or more risk haplotypes are present.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a table of results from Transmission Distortion Test, used to test association to disease. (a) depicts results from a Study Family Population; (b) depicts results from a Pediatric Population.

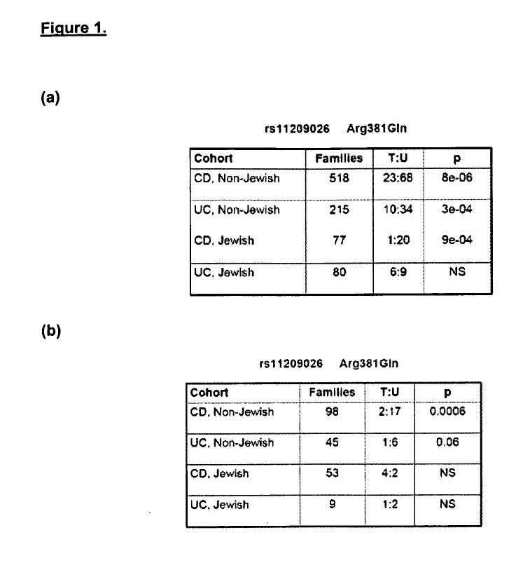

FIG. 2 depicts chromosome 1 and IL23R SNPS and positions.

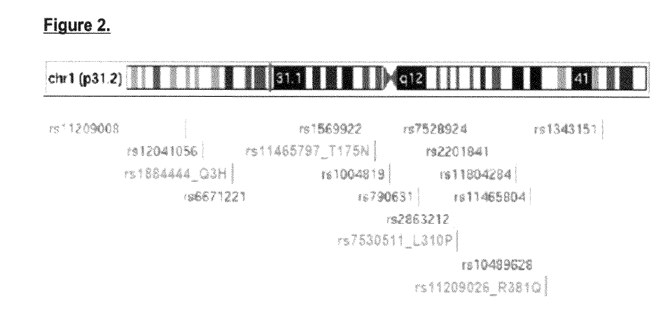

FIG. 3 depicts a graph of an example of SNPS associated with Crohn's Disease. Eight IL23R SNPS were ultimately found to be associated with Crohn's Disease and this is a graph demonstrating an example of this, comparing Crohn's Disease vs. Control for markers rs1343151 and rs11209026.

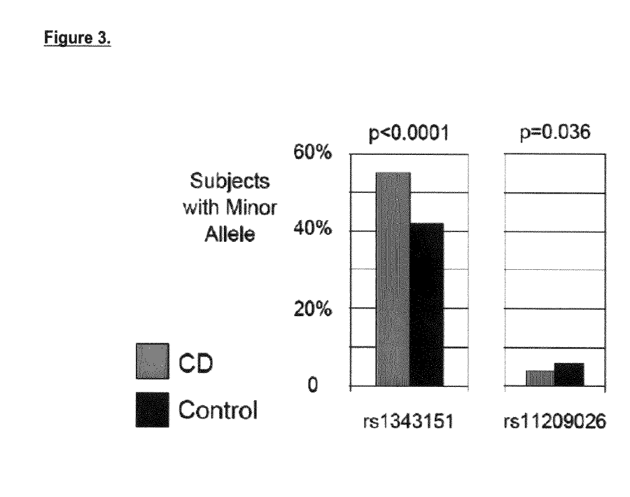

FIG. 4 depicts the SNPs, alleles, and positions of markers and three haplotype blocks observed in IL23R.

FIG. 5 depicts IL23R haplotype analysis. Block 2 is further described with corresponding haplotypes, nucleotides, and positions on chromosome.

FIG. 6 depicts IL23R haplotype analysis. Block 2 is further described, with a graph demonstrating H1 “risk” and H2 “protective” association for Crohn's Disease.

FIG. 7 depicts a chart demonstrating Crohn's Disease risk for IL23R Block 2 haplotypes.

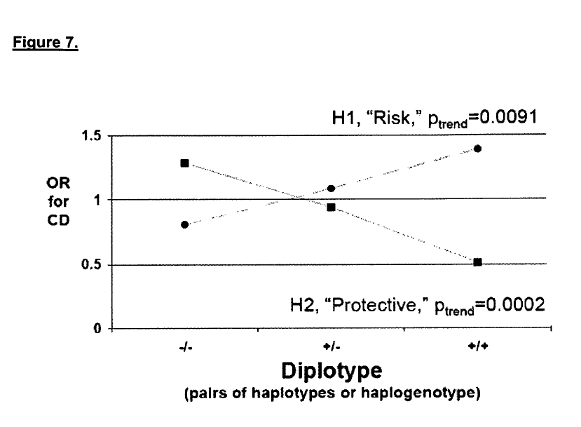

FIG. 8 depicts a chart further describing SNPs, alleles, and positions of markers and haplotypes in Block 3 of IL23R.

FIG. 9 depicts a graph further describing Block 3 of IL23R, demonstrating H1 “risk” and H2 “protective” and H6 “protective” association for Crohn's Disease.

FIG. 10 depicts a chart demonstrating Crohn's Disease risk for IL23R Block 3 haplotypes.

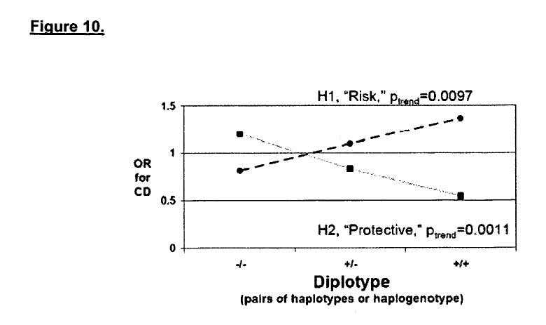

FIG. 11 depicts a chart demonstrating IL23R haplotype combinations are associated with Crohn's Disease.

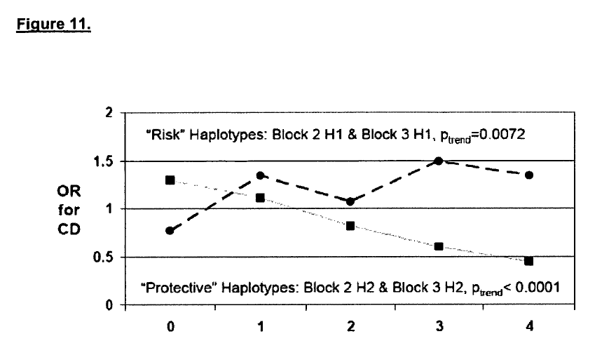

FIG. 12 depicts population attributable risk. The chart describes haplotypes of IL23R block 2, block 3, and both.

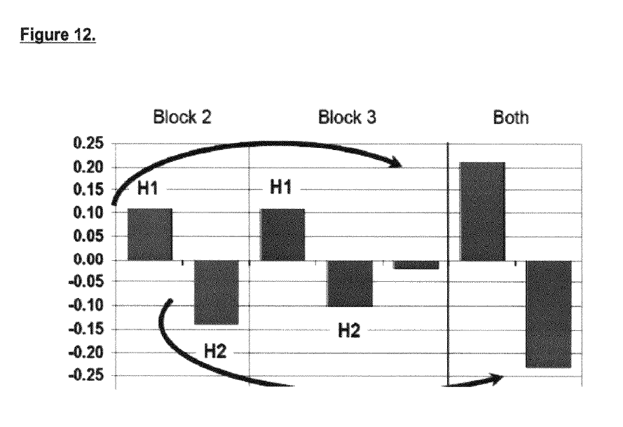

FIG. 13 depicts a chart of IL23R risk haplotypes. The chart describes both IL23R block 2 and 3 in correlation with I2 antibody expression levels.

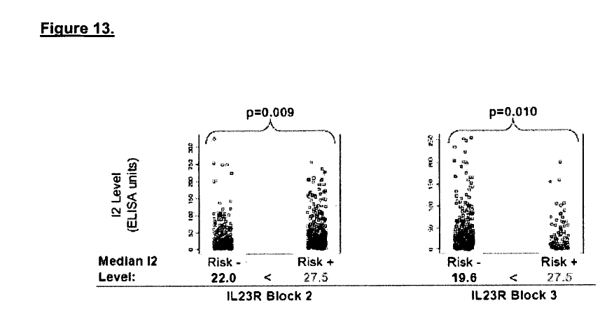

FIG. 14 depicts haplotype structure of IL17A and haplotype frequencies.

FIG. 15 depicts a chart of IL17A in non-jewish individuals with Crohn's Disease. The chart demonstrates IL17A H2 “risk” association and IL17A H4 “protective” association with Crohn's Disease.

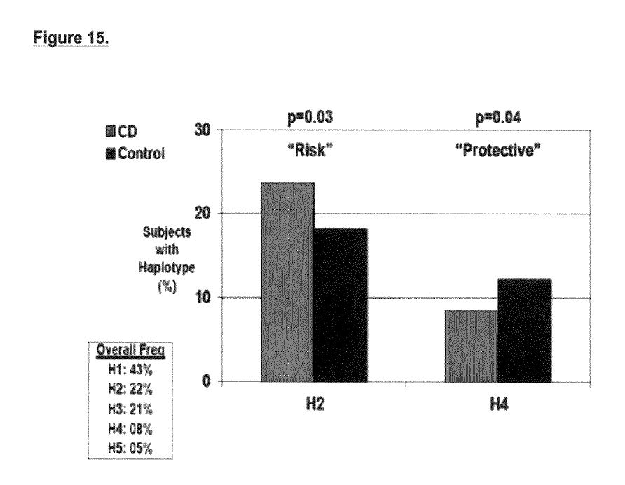

FIG. 16 depicts a chart of IL17A diplotypes in non-Jewish Crohn's Disease, with diplotype equaling pairs of haplotypes, which in turn equaling haplogenotype.

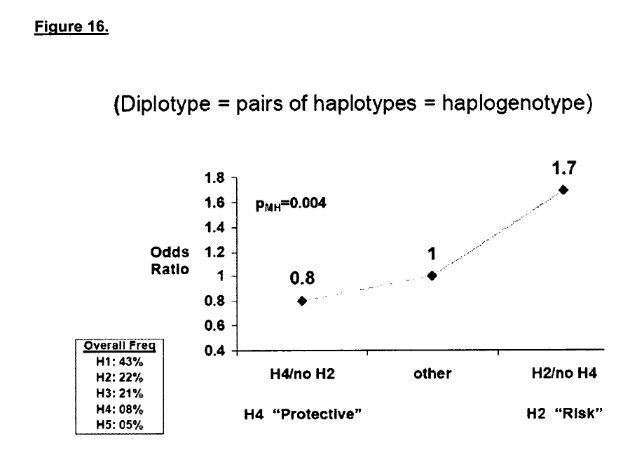

FIG. 17 depicts the haplotype structure of IL17RA and haplotype frequencies.

FIG. 18 depicts IL17RA in combined Crohn's Disease and ulcerative colitis. The chart depicts IL17RA block 2 H4 “risk” association and IL17RA block 1 H3 “protective” association with IBD.

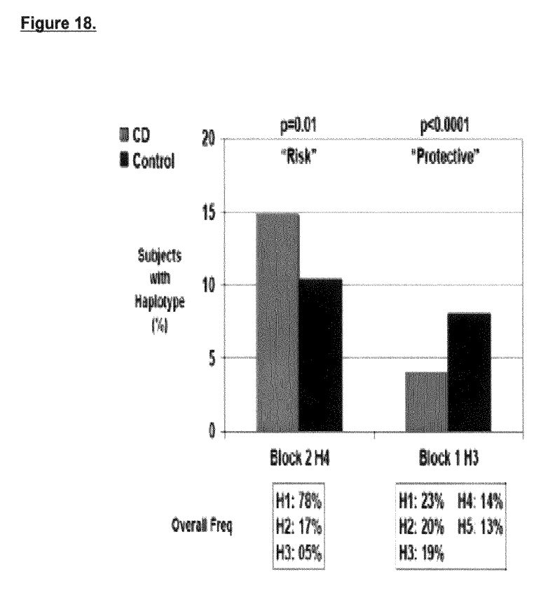

FIG. 19 depicts a chart of IL17RA haploblocks in combined Crohn's Disease and ulcerative colitis.

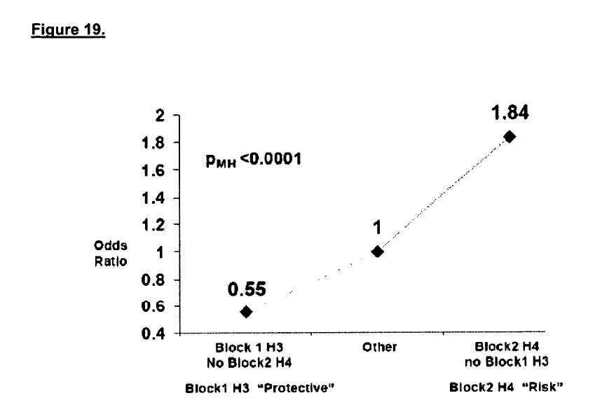

FIG. 20 depicts a graph of IL17A in Jewish and non-Jewish subgroups. The chart describes IL17A H4 “protective” and H2 “risk” association for non-Jewish Crohn's Disease patients, and IL17A H2 “protective” association for Jewish Crohn's Disease patients.

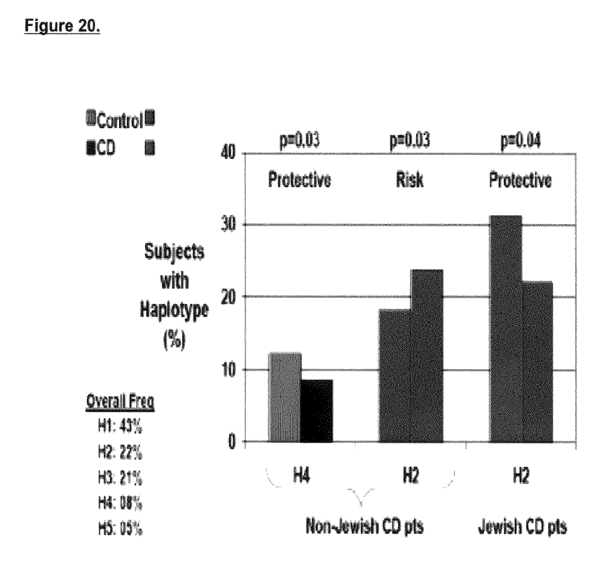

FIG. 21 depicts a chart of haplotype defined gene-gene interactions. The chart demonstrates the presence of synergy between IL23R and IL17A, and the presence of synergy between IL23R and IL17RA.

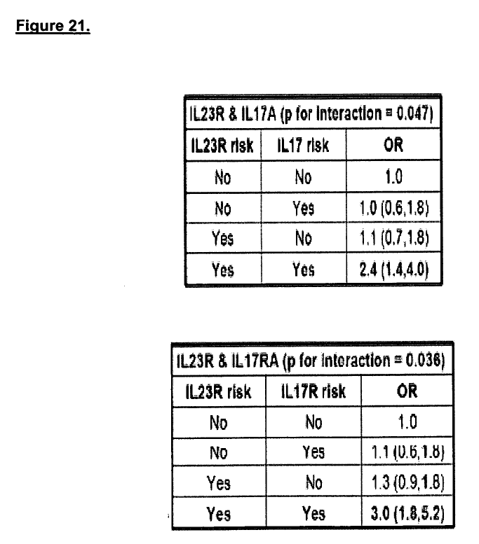

FIG. 22 depicts a chart demonstrating a lack of synergistic effect between IL17A and IL17RA in terms of gene-gene interactions.

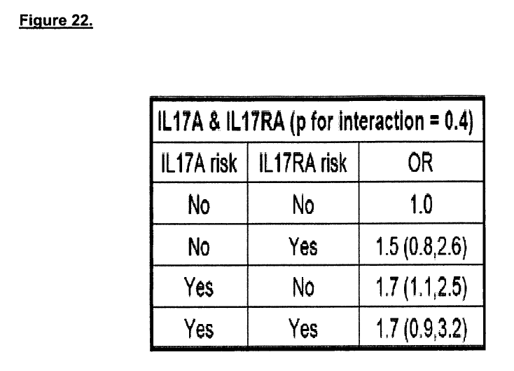

FIG. 23 depicts a chart of the combined effect of IL23R, IL17A, and IL17RA, as demonstrated by plots of no risk haplotype, one risk haplotype, two risk haplotype, and three risk haplotype.

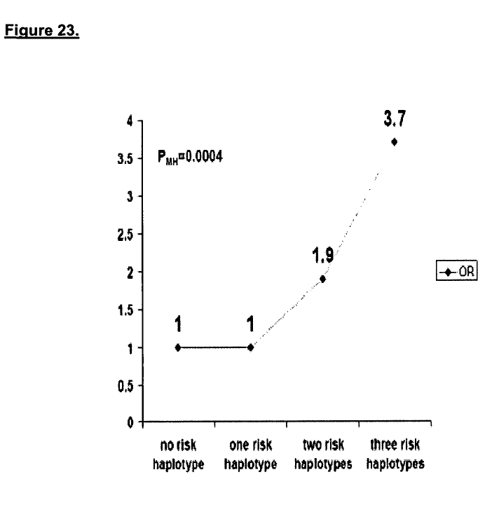

FIG. 24 depicts the IL12B haplotype structure, as well as a chart of haplotype frequency.

FIG. 25 depicts a graph of the association between IL12B haplotype and Crohn's Disease.

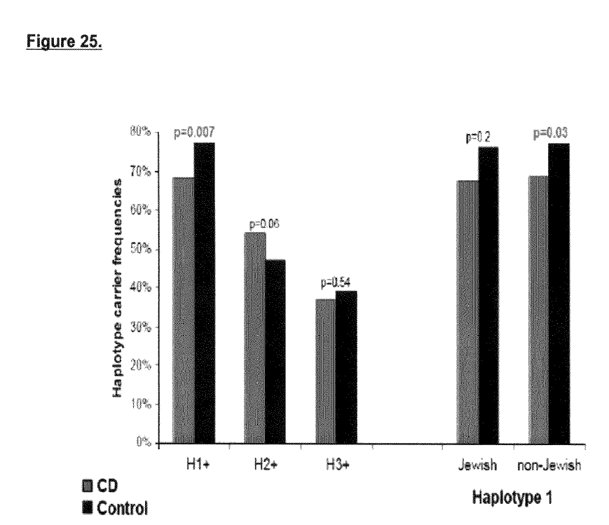

FIG. 26 depicts a graph of the association between IL12B and the presence of Anti-Cbir1.

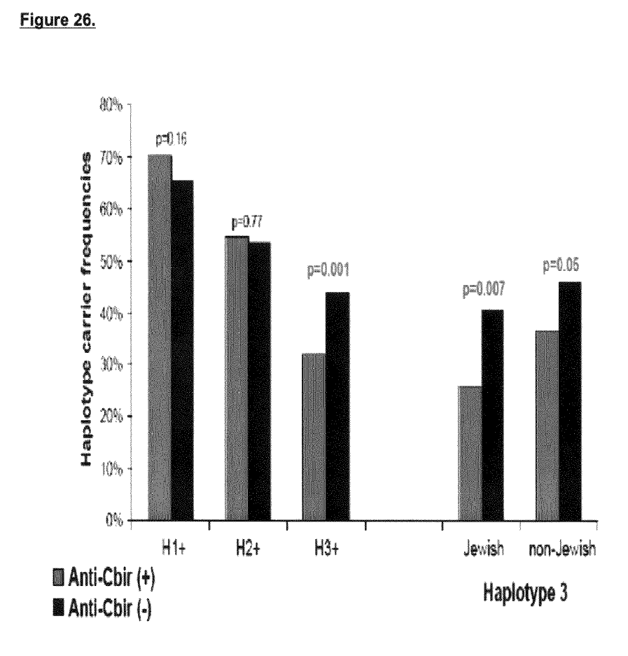

FIG. 27 depicts a graph of the association between IL12B H3 and Anti-Cbir1 level.

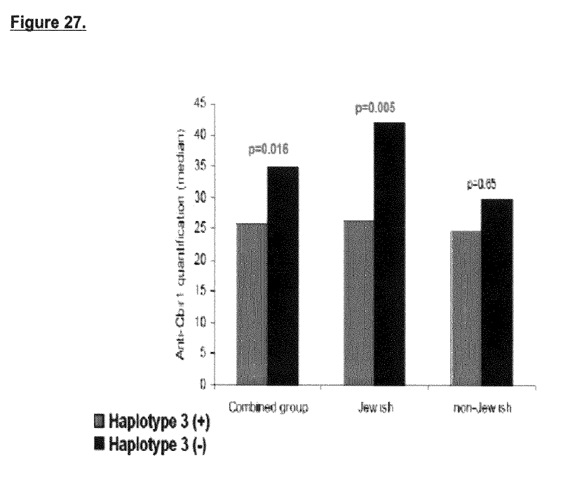

FIG. 28 depicts a chart of haplotype defined gene-gene interactions. The chart demonstrates no synergistic effects between IL12B and IL23R protective haplotypes.

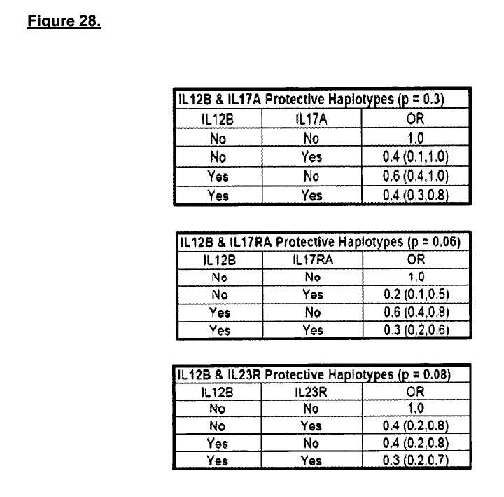

FIG. 29 depicts a chart of risk haplotype defined gene-gene interactions of IL17A, IL17RA, and IL23R.

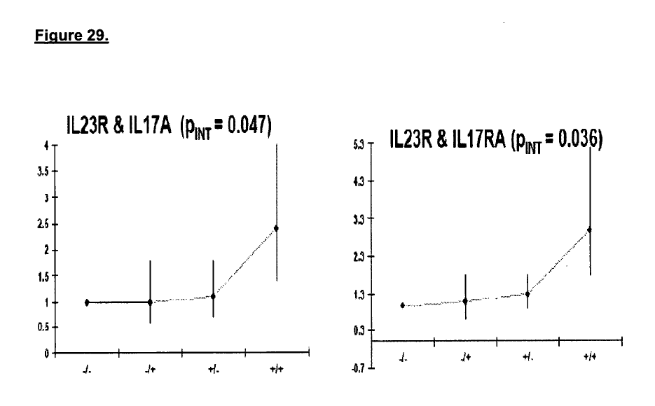

FIG. 30 depicts a chart of protective haplotype defined gene-gene interactions of IL17A, IL17RA, and IL23R with IL12B.

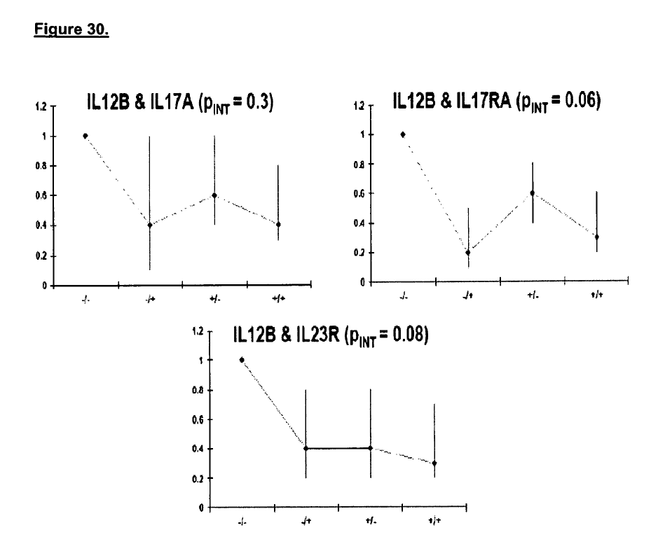

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Haplotype” as used herein refers to a set of single nucleotide polymorphisms (SNPs) on a gene or chromatid that are statistically associated.

“Protective” and “protection” as used herein refer to a decrease in susceptibility to IBD, including but not limited to CD.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As used herein, “positive seroreactivity” means a high level of expression for an antibody relative to levels that would be found in a healthy individual. For example, determining the presence of Cbir1 antibody expression means that there is a high expression level of the Cbir1 antibody relative to the levels that would be found in a healthy individual. Conversely, determining the absence of Cbir1 antibody expression means that there is a low expression level of the Cbir1 antibody relative to the levels that would be found in a diseased individual.

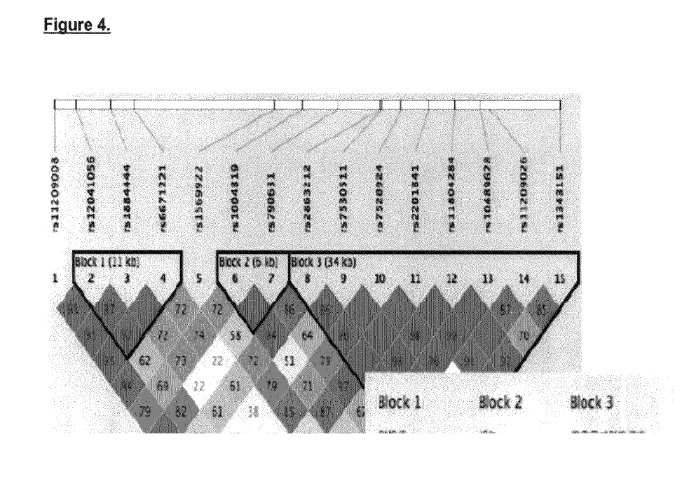

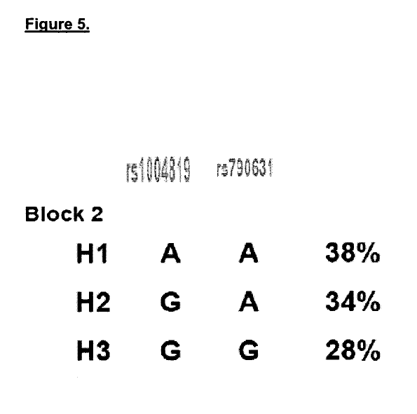

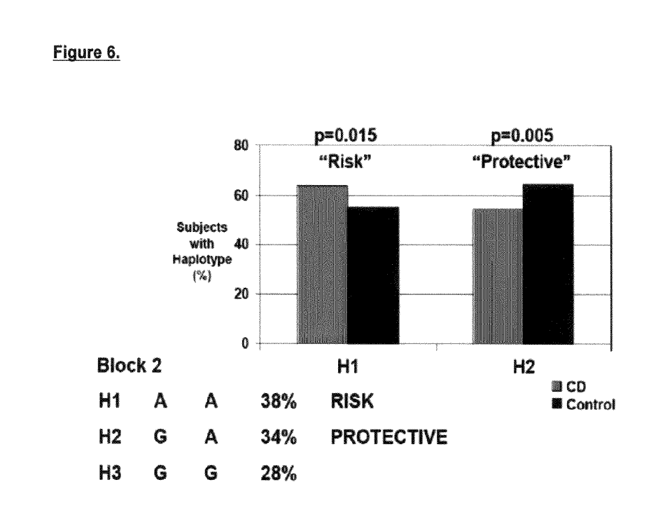

The identities of the IL23R Block 2 markers, their location on the gene and their nucleotide substitutions may be found in FIGS. 4-6.

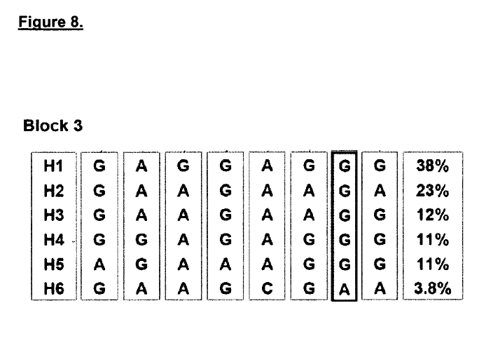

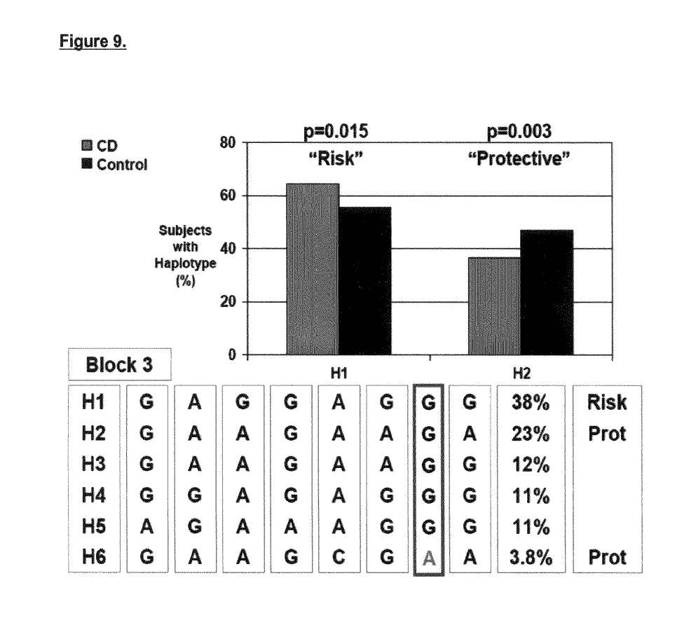

The identities of the IL23R Block 3 markers, their location on the gene and their nucleotide substitutions may be found in FIGS. 4 and 8-9.

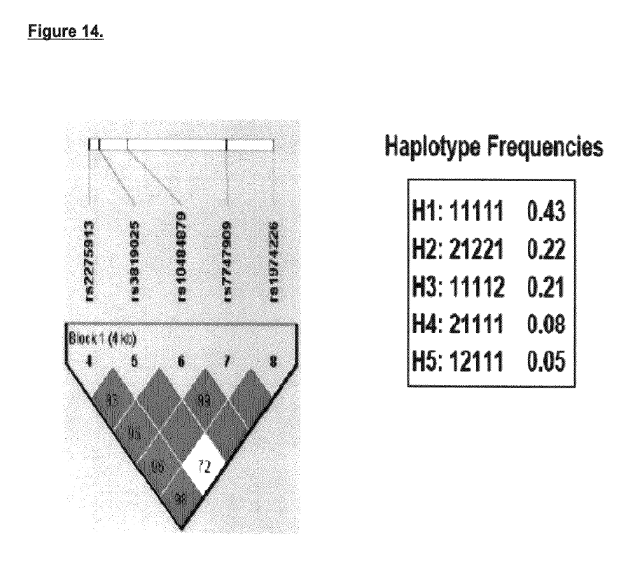

The identities of the IL17A markers, their location on the gene and their nucleotide substitutions may be found in Table 2, as well as FIG. 14.

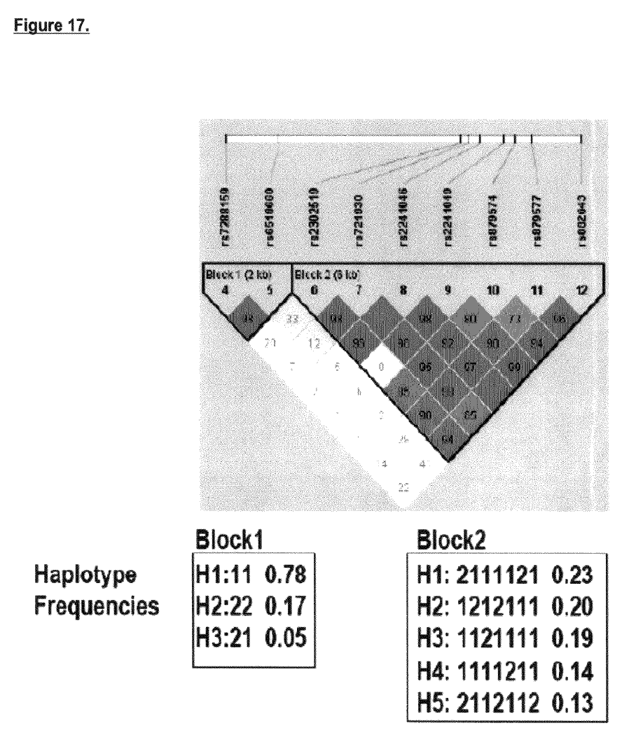

The identities of the IL17RA markers, their location on the gene and their nucleotide substitutions may be found in Table 3, as well as FIG. 17.

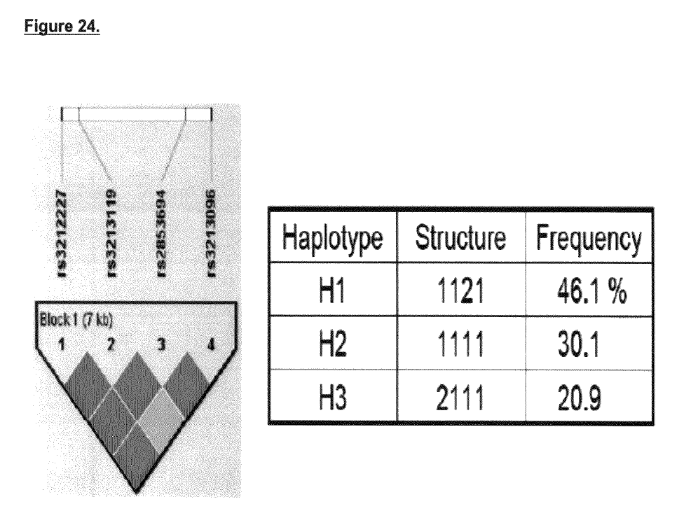

The identities of the IL12B markers, their location on the gene and their nucleotide substitutions may be found in FIG. 24.

As disclosed herein, an example of an IL23R genetic sequence is described as SEQ. ID. NO.: 1. An example of an IL23R peptide sequence is described herein as SEQ. ID. NO.: 2.

As disclosed herein, an example of an IL17A genetic sequence is described as SEQ. ID. NO.: 3. An example of an IL17A peptide sequence is described herein as SEQ. ID. NO.: 4.

As disclosed herein, an example of an IL17RA genetic sequence is described as SEQ. ID. NO.: 5. An example of an IL17RA peptide sequence is described herein as SEQ. ID. NO.: 6.

As disclosed herein, an example of an IL12B(p40) genetic sequence is described as SEQ. ID. NO.: 7. An example of an IL12B(p40) peptide sequence is described herein as SEQ. ID. NO.: 8.

Examples of the IL23R polymorphisms rs1004819, rs790631, rs2863212, rs7530511, rs7528924, rs2201841, rs11804284, rs10489628, rs11209026, and rs1343151, are also described herein as SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, and SEQ. ID. NO.: 18, respectively.

Examples of the IL17A polymorphisms rs2275913, rs3819025, rs10484879, rs7747909, and rs1974226, are also described herein as SEQ. ID. NO.: 19, SEQ. ID. NO.: 20, SEQ. ID. NO.: 21, SEQ. ID. NO.: 22, and SEQ. ID. NO.: 23, respectively.

Examples of the IL17RA polymorphisms rs7288159, rs6518660, rs2302519, rs721930, rs2241046, rs2241049, rs879574, rs879577, and rs882643, are also described herein as SEQ. ID. NO.: 24, SEQ. ID. NO.: 25, SEQ. ID. NO.: 26, SEQ. ID. NO.: 27, SEQ. ID. NO.: 28, SEQ. ID. NO.: 29, SEQ. ID. NO.: 30, SEQ. ID. NO.: 31, and SEQ. ID. NO.: 32, respectively.

Examples of the IL12(p40) polymorphisms rs3212227, rs3213119, rs2853694, and rs3213096, are also described herein as SEQ. ID. NO.: 33, SEQ. ID. NO.: 34, SEQ. ID. NO.: 35, and SEQ. ID. NO.: 36, respectively.

As used herein, an “interaction” of genetic variants for conferring susceptibility to a disease is defined as an additive effect for the variants' association with susceptibility to the disease, so that the genetic variants are not independently associated with the disease. For example, in the case of an interaction determined to exist between two risk haplotypes, the presence of the two risk haplotypes would be determined to confer a greater susceptibility to the disease than would the presence of only one or none of the risk haplotypes.

As known to one of ordinary skill in the art, there are presently various treatments and therapies available for those diagnosed with Inflammatory Bowel Disease, including but not limited to surgery, anti-inflammatory medications, steroids, and immunosuppressants.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to I2, OmpC, and Cbir. The detection of protective and risk SNPs and/or haplotypes may be used to identify at risk individuals, predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and ulcerative colitis. Other embodiments provide for methods of treating inflammatory bowel disease including but not limited to Crohn's Disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

### IL23 Receptor (IL23R) Gene Protects Against Pediatric Crohn's Disease

As disclosed herein, the inventors examined the association of IL23R with susceptibility to ulcerative colitis (UC) and CD in pediatric patients. DNA was collected from 610 subjects (152 CD trios, 52 UC trios). Both parents and the affected child were genotyped for the protective R381Q SNP (rs11209026) of the IL23R gene and 4 variants of the CARD15 gene (SNP5, SNP8, SNP12, SNP13) using Taqman technology. The transmission disequilibrium test (TDT) was used to test association to disease using GENEHUNTER 2.0.

As further disclosed herein, the rare allele of R381Q SNP was present in 5.3% of CD and 5.9% UC probands. CARD15 frequency (any variant) was 35% (CD) and 11% (UC). Similar frequencies were observed for parents for both genes. The IL23R allele was negatively associated with IBD: the R381Q SNP was undertransmitted in children with IBD (8 transmitted (T) vs. 27 untransmitted (UT); p=0.001) (See Table 1). This association was significant for all CD patients (6 T vs. 19 UT; p=0.009), especially for non-Jewish CD patients (2 T vs. 17 UT; p=0.0006). TDT showed a borderline association for UC (T 2 vs. 8 UT; p=0.06). As expected, CARD15 was associated with CD in children by the TDT: (63 T vs. 30 UT p=0.0006), but not with UC.

TABLE 1

| | IBD | | | CD | | | UC | | |
|---|---|---|---|---|---|---|---|---|---|
| IL23R | | | P | | | P | | | P |
| rare allele | T | UT | VALUE | T | UT | VALUE | T | UT | VALUE |
| R381Q SNP | 8 | 27 | 0.001 | 6 | 19 | 0.009 | 2 | 8 | P = 0.06 |

T = Transmitted
UT = Undertransmitted

As further disclosed herein, the CARD15 association acted as a control in this study, with the observed association with CARD15 demonstrating that applying the TDT to this pediatric cohort will be useful in further gene finding for IBD. The protective IL23R R381Q variant was particularly associated with CD in non-Jewish children.

In one embodiment, the present invention provides methods of diagnosing and/or predicting protection against IBD in an individual by determining the presence or absence of the protective R381Q SNP (rs11209026) of the IL23R gene. In another embodiment, the IBD comprises Crohn's Disease. In another embodiment, the IBD comprises ulcerative colitis. In another embodiment, the individual is a pediatric. In another embodiment, the individual is non-Jewish.

### High Frequency IL23R Haplotypes Explain A High Percentage Risk

As disclosed herein, the inventors studied the association of IL23R haplotypes with CD and associated serotypes. CD subjects (n=763) and ethnically-matched controls (254) were genotyped for 20 single-nucleotide polymorphisms (SNPs) using Illumina and TaqMan MGB technologies. SNPs were selected to tag Caucasian haplotypes using HapMap data. Serum expression of antibodies was determined by ELISA. Presence of disease, IL23R genotype, and serum antibodies were each determined blinded. Haplotypes were determined with PHASE v2; associations with disease were tested by chi-square and to antibody expression by Wilcoxon.

As further disclosed herein, three haplotype blocks were observed in the IL23R gene. Block 3 spans the protective SNP R381Q. Associations with both a “risk” haplotype and a “protective” haplotype were observed in Blocks 2 and 3 (Block 2: Risk, 64% in CD, 55% in controls, p=0.015; Protective, 54% in CD, 65% in controls, p=0.005; Block 3: Risk, 64% CD, 56% controls, p=0.015; Protective, 37% CD, 47% controls, p=0.003). Block 2 risk and Block 3 risk are additive for increased risk (ptrend=0.0072) and Block 2 protective and Block 3 protective are additive for decreased risk (ptrend<0.0001). Population attributable risk (PAR) for Block 2 and Block 3 risk is ˜10-20% and is much greater than the PAR for the low frequency R381Q (˜2%). The Block 3 risk haplotype was associated with increased serum expression of anti-I2 antibody (median I2 level for presence of risk haplotype 27.5 compared with 19.6 for absence of risk haplotype, p=0.01).

As further disclosed herein, IL23R risk haplotypes confer marked, additional CD risks compared with the functional, protective SNP IL23R R381Q. IL23R therefore accounts for a substantial increase in CD risk. Furthermore, IL23R haplotypes are associated with serum expression of antibody to 12, a Pseudomonas related antigen. Subjects with these haplotypes will be important for studying IL23R function.

In one embodiment, the present invention provides methods of diagnosing and/or predicting susceptibility to Crohn's Disease in an individual by determining the presence or absence of IL23R risk haplotype H1 of Block 2. In another embodiment, the present invention provides methods of diagnosing and/or predicting protection against Crohn's Disease in an individual by determining the presence or absence of IL23R protective haplotype H2 in Block 2.

In another embodiment, the present invention provides methods of treatment of Crohn's Disease in an individual by determining the presence or absence of IL23R risk haplotype H1 of Block 2, and then treating the Crohn's Disease.

In one embodiment, the present invention provides methods of diagnosing and/or predicting susceptibility to Crohn's Disease in an individual by determining the presence or absence of IL23R risk haplotype H1 of Block 3. In another embodiment, the present invention provides methods of diagnosing and/or predicting protection against Crohn's Disease in an individual by determining the presence or absence of IL23R protective haplotype H2 in Block 3. In another embodiment, the present invention provides methods of diagnosing and/or predicting protection against Crohn's Disease in an individual by determining the presence or absence of IL23R protective haplotype H6 in Block 3.

In another embodiment, the present invention provides methods of treatment of Crohn's Disease in an individual by determining the presence or absence of IL23R risk haplotype H1 of Block 3, and then treating the Crohn's Disease.

In one embodiment, the present invention provides methods of diagnosing and/or predicting susceptibility to Crohn's Disease in an individual by determining the presence or absence of IL23R risk haplotype H1 of Block 2 and/or IL23R risk haplotype H1 of Block 3.

In another embodiment, the present invention provides methods of treatment of Crohn's Disease in an individual by determining the presence or absence of IL23R risk haplotype H1 of Block 2 and/or IL23R risk haplotype H1 of Block 3, followed by administering treatment of the Crohn's Disease.

In one embodiment, the present invention provides methods of diagnosing and/or predicting protection against Crohn's Disease in an individual by determining the presence or absence of IL23R protective haplotype H2 in Block 2, IL23R protective haplotype H2 in Block 3, and/or IL23R protective haplotype H6 in Block 3.

In one embodiment, the present invention provides methods of diagnosing and/or predicting susceptibility to Crohn's Disease in an individual by determining the presence or absence of IL23R risk haplotype H1 of Block 3 and increased serum expression of anti-12 antibody.

In another embodiment, the present invention provides methods of treatment of Crohn's Disease in an individual by determining the presence or absence of IL23R risk haplotype H1 of Block 3 and increased serum expression of anti-12 antibody, followed by administering treatment for the Crohn's Disease.

### Association Between IL17A and IL17RA Genes and Inflammatory Bowel Disease

As disclosed herein, IL17A is produced by TH17 CD4+ T cells, and in some mouse models of colitis, IL17A is responsible for mucosal inflammation. Its role in human IBD is not yet known. IL17RA is a ubiquitously expressed receptor that is essential for IL17A biologic activity. The inventors determined whether IL17A and/or IL17RA genes are associated with IBD. SNPs were selected to tag common Caucasian haplotypes in IL17A (#3605) and IL17RA (#23765) and genotyped in 763 Crohn's disease (CD), 351 ulcerative colitis (UC) and 254 controls using Illumina technology. Analysis was first done in the total sample, and then Haploview 3.3. Individual haplotypes were obtained by PHASE v2 and ordered by frequency (See Tables 2 and 3).

TABLE 2
Haplotype of IL17A (1: rare allele)

| SNP | H1 | H2 | H3 | H4 | H5 |
|---|---|---|---|---|---|
| rs2275913 | 2 | 1 | 2 | 1 | 2 |
| rs3819025 | 2 | 2 | 2 | 2 | 1 |
| rs10484879 | 2 | 1 | 2 | 2 | 2 |
| rs7747909 | 2 | 1 | 2 | 2 | 2 |
| rs1974226 | 2 | 2 | 1 | 2 | 2 |

TABLE 3
Haplotype of IL17RA (1: rare allele)

| SNP | H1 | H2 | H3 | H4 | H5 |
|---|---|---|---|---|---|
| Block1: | | | | | |
| rs7288159 | 2 | 1 | 1 | | |
| rs6518660 | 2 | 1 | 2 | | |
| Block2: | | | | | |
| rs2302519 | 1 | 2 | 2 | 2 | 1 |
| rs721930 | 2 | 1 | 2 | 2 | 2 |
| rs2241046 | 2 | 2 | 1 | 2 | 2 |
| rs2241049 | 2 | 1 | 2 | 2 | 1 |
| rs879574 | 2 | 2 | 2 | 1 | 2 |
| rs879577 | 1 | 2 | 2 | 2 | 2 |
| rs882643 | 2 | 2 | 2 | 2 | 1 |

As further disclosed herein, two major haplotypes (H2 and H4) of IL17A were associated with CD. In non-Jews, CD patients had a higher frequency of H2 (23.7% vs. 18.2%, p=0.03) and a lower frequency of H4 (8.5% vs. 12.3%, P=0.03) when compared with controls; however, an opposite trend was found in the Jewish population for H2 (22.1% vs. 31.4%, P=0.04). Diplotype (i.e. haplogenotype) analysis for IL17A in non-Jews showed a significant trend for odds ratio (OR): H4/no H2 (OR 0.8), other combinations (OR 1), H2/no H4 (OR 1.7, P Mantel-Hanzel=0.004). IL17RA. Two haplotype blocks were identified for IL-17RA. In the total sample, haplotype 3 (H3) in block 1 was negatively associated with both CD and UC when compared with controls (4.0% vs. 8.1%, P<0.0001). In block 2, H4 was positively associated with IBD (14.8% vs. 10.4%, P=0.01). The results were similar in Jews and non-Jews. The combined analysis for the two blocks of IL17RA also displayed a significant trend for increased OR: H3 block 1/no H4 block 2 (OR 0.55), other, (OR 1), H4 no H3 (OR: 1.84, P Mantel-Hanzel <0.0001).

As further disclosed herein, IL17A appears to be an ethnic specific gene for CD, and IL17RA is a gene associated with both CD and UC. This cytokine/receptor pair is important in the pathogenesis of a subtype of CD.

In one embodiment, the present invention provides methods of diagnosing and/or predicting susceptibility to Crohn's Disease in a non-Jewish individual by determining the presence or absence of a high frequency of IL17A haplotype H2 and a lower frequency of IL17A haplotype H4. In another embodiment, the present invention provides methods of diagnosing and/or predicting susceptibility to Crohn's Disease in a Jewish individual by determining the presence or absence of a low frequency of IL17A haplotype H2.

In another embodiment, the present invention provides methods of treatment for Crohn's Disease in a non-Jewish individual by determining the presence or absence of a high frequency of IL17A haplotype H2 and a lower frequency of IL17A haplotype H4, followed by administering treatment for the Crohn's Disease. In another embodiment, the present invention provides methods of treatment for Crohn's Disease in a Jewish individual by determining the presence or absence of a low frequency of IL17A haplotype H2, followed by administering treatment for the Crohn's Disease.

In one embodiment, the present invention provides methods of diagnosing and/or predicting susceptibility to Inflammatory Bowel Disease in an individual by determining the presence or absence of a low frequency of IL17RA haplotype H3 and a high frequency of IL17RA haplotype H4.

In another embodiment, the present invention provides methods of treatment for Inflammatory Bowel Disease in an individual by determining the presence or absence of a low frequency of IL17RA haplotype H3 and a high frequency of IL17RA haplotype H4, and then administering treatment for the Crohn's Disease.

### An Interaction Between IL-23R and IL-17A and Between IL-23R and IL-17RA Haplotypes is Necessary for Susceptibility to Crohn's Disease

As disclosed herein, the inventors determined whether an interaction exists between IL-23R and IL-17A/IL-17RA genetic variants for conferring susceptibility to CD development. SNPs were selected to tag common haplotypes and genotyped in 763 CD and 254 controls using Illumina technology. Haplotype blocks were constructed using Haploview 3.3. Analysis was done in the total sample first, and then in Jewish and non-Jewish subjects separately. Analysis for gene interaction was performed using the Breslow-Day test.

As used herein, an “interaction” of genetic variants for conferring susceptibility to a disease is defined as an additive effect for the variants' association with susceptibility to the disease, so that the genetic variants are not independently associated with the disease. For example, in the case of an interaction determined to exist between two risk haplotypes of a Crohn's Disease, the presence of the two risk haplotypes would be determined to confer a greater susceptibility to the Crohn's Disease than would the presence of only one or none of the risk haplotypes.

As further disclosed herein, two IL23R risk haplotypes were identified (IL23R block 3 H1 and block 2 H1) and one each for IL17A (IL17A H2) and IL17RA (IL17RA H4) to confer increased risk for CD. In terms of an IL23R and IL17A interaction, while the risk haplotype for each gene contributed susceptibility individually, there was no increased risk for disease if either of the two genes' risk haplotypes were absent. IL-23R absent/IL-17A risk (OR 1.04, p=NS); IL-23R risk/IL-17A absent (OR 1.1, p=NS); however, the combination of the risk haplotypes from IL23R with the risk haplotype from IL17A dramatically increased risk for CD (30% in non-Jewish CD vs. 16% of controls, OR 2.4; p for interaction 0.047). In terms of an IL23R and IL17RA interaction, IL23R absent/IL17RA risk (OR 1.1, p=NS); IL23R risk/IL17RA absent (OR 1.3, p=NS): i.e. no increased risk if a risk haplotype was absent. Yet again the combination dramatically increased risk in the total CD sample (OR 3.0, p for interaction 0.036). In terms of an IL17A and IL17RA interaction, in contrast, the inventors found no interaction between the IL17A and the IL17RA haplotypes in non-Jewish CD (P=0.4). When all three haplotypes were examined sequentially for interaction, the OR for CD in the non-Jewish population increased from 1 when neither haplotype was present to 3.7 (CI 1.3-10.1, P_{Mantel-Hanzel}=0.0004) (See Table 4).

TABLE 4

| IL23R risk | IL17RA risk | IL17A risk | OR (CI) | P value |
|---|---|---|---|---|
| No | No | No | 1 | 0.004 |
| * | * | * | 1.0 (0.7, 1.6) | |
| ** | ** | ** | 1.9 (1.1, 3.2) | |
| Yes | Yes | Yes | 3.7 (1.3, 10.1) | |

*One risk Haplotype present (of either IL23R, IL17RA or IL-17A),
**Two risk Haplotypes present (of either IL23R, IL17RA or IL-17A)

As further disclosed herein, the data demonstrates the multiple and likely complex interactions between the individual components of the IL-23/IL-17 axis, which therefore appear to be playing a significant role in CD mucosal inflammation.

In one embodiment, the present invention provides methods of diagnosing and/or predicting susceptibility for Crohn's Disease in an individual by determining the presence or absence of one or more risk haplotypes at the IL-23R locus and/or the IL-17A locus. In another embodiment, the present invention provides methods of treatment of Crohn's Disease in an individual by determining the presence or absence of one or more risk haplotypes as the IL23R locus and/or the IL-17A locus, and then administering a treatment for the Crohn's Disease.

In one embodiment, the present invention provides methods of diagnosing and/or predicting susceptibility to Crohn's Disease in an individual by determining the presence or absence of IL23R risk haplotype block 3 H1, IL23R risk haplotype block 2 H1, and/or IL17A risk haplotype H2. In another embodiment, the present invention provides methods of treatment of Crohn's Disease in an individual by determining the presence or absence of IL23R risk haplotype block 3 H1, IL23R risk haplotype block 2 H1, and/or I17A risk haplotype H2.

As mentioned above, the identities of the IL23R Block 2 markers, their location on the gene and their nucleotide substitutions may be found in FIGS. 4-6; the identities of the IL23R Block 3 markers, their location on the gene and their nucleotide substitutions may be found in FIGS. 4 and 8-9; the identities of the IL17A markers, their location on the gene and their nucleotide substitutions may be found in Table 2, as well as FIG. 14.

In one embodiment, the present invention provides methods of diagnosing and/or predicting susceptibility for Crohn's Disease in an individual by determining the presence or absence of one or more risk haplotypes at the IL-23R locus and/or IL-17RA locus. In another embodiment, the present invention provides methods of treatment of Crohn's Disease in an individual by determining the presence or absence of one or more risk haplotypes at the IL-23R locus and/or IL-17RA locus, and then administering a treatment for the Crohn's Disease.

In one embodiment, the present invention provides methods of diagnosing and/or predicting susceptibility to Crohn's Disease in an individual by determining the presence or absence of IL23R risk haplotype block 3 H1, IL23R risk haplotype block 2 H1, and/or IL17RA risk haplotype H4. In another embodiment, the present invention provides methods of treatment of Crohn's Disease in an individual by determining the presence or absence of IL23R risk haplotype block 3 H1, IL23R risk haplotype block 2 H1, and/or IL17RA risk haplotype H4, and then administering a treatment for the Crohn's Disease.

As mentioned above, the identities of the IL17RA markers, their location on the gene and their nucleotide substitutions may be found in Table 3, as well as FIG. 17.

### Different Haplotypes of the IL12B(p40) Gene are Associated with Clinical Crohn's Disease and with Crohn's Disease Patients Expressing Cbir1 Antibodies, Respectively

As disclosed herein, the IL12B gene codes for the p40 subunit shared in common by IL12 and IL23, key cytokines that bridge innate and Th1/Th17 adaptive immune responses. CD has been associated with increased secretion of IL12 and IL23, and treatment with p40 antibody has been effective in certain CD patients. The inventors have previously shown that the antibody response to microbial antigens defines different groups of IBD patients, including those with complicated disease.

As further disclosed herein, the inventors investigated IL12B associations with CD and antibody expression. Four IL12B SNPs: rs3212227 (previously associated with autoimmune disease), F298V, rs2853694 (intron 4), and 133V were genotyped by Illumina GoldenGate Assay in 763 CD patients, and 254 controls. Serum antimicrobial antigens were measured by ELISA. Chi-square was used to test for association of haplotypes with disease and presence of antibody. One haplotype block was found by Haploview 3.3. Individual haplotypes were obtained by PHASE and ordered by frequency. Among three common haplotypes, H1 (H1:2212) was negatively associated with CD, i.e. protective (CD vs control: 68.3% vs 77.2%, p=0.007), with similar direction in both Jews and non-Jews. The inventors also observed an association between H3 (H3:1222) and anti-Cbir1 expression in these CD patients, in that H3 frequency was significantly lower in the patients who were anti-Cbir1 positive (31.8% vs 43.9%, p=0.001). This association was again observed in both Jews and non-Jews.

As further disclosed herein, the inventors have identified one IL12B gene haplotype protective for clinical CD and a different protective haplotype in CD patients who expressed antibody to CBir1. These results support the concept that IL12B variants, and therefore, IL12 and/or IL23 are involved in the overall susceptibility to CD as well as the subtype of CD patients defined by anti-CBir1 expression.

In one embodiment, the present invention provides methods of diagnosing and/or predicting protection against Crohn's Disease in an individual by determining the presence or absence of H1. In another embodiment, the present invention provides methods of treatment of Crohn's Disease in an individual by determining the presence or absence of H1, and then administering a treatment for the Crohn's Disease.

In one embodiment, the present invention provides methods of diagnosing and/or predicting protection against Crohn's Disease in an individual by determining the presence or absence of H3 with a lack of anti-Cbir1 expression. In another embodiment, the present invention provides methods of treatment of Crohn's Disease in an individual by determining the presence or absence of H3 with a lack of anti-Cbir1 expression, and then administering a treatment for the Crohn's Disease.

### Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of an IL23R variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### High Frequency IL23R Haplotypes Explain A High Percentage Risk

The inventors studied the association of IL23R haplotypes with CD and associated serotypes. CD subjects (n=763) and ethnically-matched controls (254) were genotyped for 20 single-nucleotide polymorphisms (SNPs) using Illumina and TaqMan MGB technologies. SNPs were selected to tag Caucasian haplotypes using HapMap data. Serum expression of antibodies was determined by ELISA. Presence of disease, IL23R genotype, and serum antibodies were each determined blinded. Haplotypes were determined with PHASE v2; associations with disease were tested by chi-square and to antibody expression by Wilcoxon.

Three haplotype blocks were observed in the IL23R gene. Block 3 spans the protective SNP R381Q. Associations with both a “risk” haplotype and a “protective” haplotype were observed in Blocks 2 and 3 (Block 2: Risk, 64% in CD, 55% in controls, p=0.015; Protective, 54% in CD, 65% in controls, p=0.005; Block 3: Risk, 64% CD, 56% controls, p=0.015; Protective, 37% CD, 47% controls, p=0.003). Block 2 risk and Block 3 risk are additive for increased risk (ptrend=0.0072) and Block 2 protective and Block 3 protective are additive for decreased risk (ptrend<0.0001). Population attributable risk (PAR) for Block 2 and Block 3 risk is ˜10-20% and is much greater than the PAR for the low frequency R381Q (˜2%). The Block 3 risk haplotype was associated with increased serum expression of anti-12 antibody (median 12 level for presence of risk haplotype 27.5 compared with 19.6 for absence of risk haplotype, p=0.01).

Thus, IL23R risk haplotypes confer marked, additional CD risks compared with the functional, protective SNP IL21R R381Q. IL23R therefore accounts for a substantial increase in CD risk. Furthermore, IL23R haplotypes are associated with serum expression of antibody to 12, a Pseudomonas related antigen. Subjects with these haplotypes will be important for studying IL23R function. These observations increase the relative importance of this gene in the etiology of CD.

### Example 2

### IL23 Receptor (IL23R) Gene Protects Against Pediatric Crohn's Disease

IL23R has recently been found to be associated with small bowel Crohn's disease (CD) in a large whole genome association study and the rare allele of the R381Q SNP conferred protection against CD. In the IL10-knockout mouse model of colitis, IL23R has been demonstrated to play a role in intestinal inflammation. It is unknown whether IL23R is associated with IBD in children.

The inventors examined the association of IL23R with susceptibility to ulcerative colitis (UC) and CD in pediatric patients. DNA was collected from 610 subjects (152 CD trios, 52 UC trios). Both parents and the affected child were genotyped for the protective R381Q SNP (rs11209026) of the IL23R gene and 4 variants of the CARD15 gene (SNP5, SNP8, SNP12, SNP13) using Taqman technology. The transmission disequilibrium test (TDT) was used to test association to disease using GENEHUNTER 2.0.

The rare allele of R381Q SNP was present in 5.3% of CD and 5.9% UC probands. CARD15 frequency (any variant) was 35% (CD) and 11% (UC). Similar frequencies were observed for parents for both genes. The IL23R allele was negatively associated with IBD: the R381Q SNP was undertransmitted in children with IBD (8 transmitted (T) vs. 27 untransmitted (UT); p=0.001). This association was significant for all CD patients (6 T vs. 19 UT; p=0.009), especially for non-Jewish CD patients (2 T vs. 17 UT; p=0.0006). TDT showed a borderline association for UC (T 2 vs. 8 UT; p=0.06). As expected, CARD15 was associated with CD in children by the TDT: (63 T vs. 30 UT p=0.0006), but not with UC.

Thus, the CARD15 association acted as a control in this study: the observed association with CARD15 demonstrated that applying the TDT to this pediatric cohort will be useful in further gene finding for IBD. The protective IL23R R381Q variant was particularly associated with CD in non-Jewish children. Thus, the initial whole genome association study based on ileal CD in adults has been extended to the pediatric population and beyond small bowel CD.

### Example 3

### Different Haplotypes of the IL12B(p40) Gene are Associated with Clinical Crohn's Disease (CD) and with CD Patients Expressing Cbir1 Antibodies, Respectively

The inventors investigated IL12B associations with CD and antibody expression. Four IL12B SNPs: rs3212227 (previously associated with autoimmune disease), F298V, rs2853694 (intron 4), and 133V were genotyped by Illumina GoldenGate Assay in 763 CD patients, and 254 controls. Serum antimicrobial antigens were measured by ELISA. Chi-square was used to test for association of haplotypes with disease and presence of antibody.

One haplotype block was found by Haploview 3.3. Individual haplotypes were obtained by PHASE and ordered by frequency. Among three common haplotypes, haplotype 1 (H1:2212) was negatively associated with CD, i.e. protective (CD vs control: 68.3% vs 77.2%, p=0.007), with similar direction in both Jews and non-Jews. The inventors also observed an association between haplotype 3 (H3:1222) and anti-Cbir1 expression in these CD patients, in that H3 frequency was significantly lower in the patients who were anti-Cbir1 positive (31.8% vs 43.9%, p=0.001). This association was again observed in both Jews and non-Jews.

The inventors have identified one IL12B gene haplotype protective for clinical CD and a different protective haplotype in CD patients who expressed antibody to CBir1. These results support the concept that IL12B variants, and therefore, IL12 and/or IL23 are involved in the overall susceptibility to CD as well as the subtype of CD patients defined by anti-CBir1 expression.

### Example 4

### Association Between IL 17A and IL 17RA Genes and Inflammatory Bowel Disease

The inventors determined whether IL17A and/or IL17RA genes are associated with IBD. SNPs were selected to tag common Caucasian haplotypes in IL17A (#3605) and IL17RA (#23765) and genotyped in 763 Crohn's disease (CD), 351 ulcerative colitis (UC) and 254 controls using Illumina technology. Analysis was first done in the total sample, and then Haploview 3.3. Individual haplotypes were obtained by PHASE v2 and ordered by frequency.

Two major haplotypes (H2 and H4) of IL17A were associated with CD. In non-Jews, CD patients had a higher frequency of H2 (23.7% vs. 18.2%, p=0.03) and a lower frequency of H4 (8.5% vs. 12.3%, P=0.03) when compared with controls; however, an opposite trend was found in the Jewish population for H2 (22.1% vs. 31.4%, P=0.04). Diplotype (i.e. haplogenotype) analysis for IL17A in non-Jews showed a significant trend for odds ratio (OR): H4/no H2 (OR 0.8), other combinations (OR 1), H2/no H4 (OR 1.7, P Mantel-Hanzel=0.004). IL17RA. Two haplotype blocks were identified for IL-17RA. In the total sample, haplotype 3 (H3) in block 1 was negatively associated with both CD and UC when compared with controls (4.0% vs. 8.1%, P<0.0001). In block 2, H4 was positively associated with IBD (14.8% vs. 10.4%, P=0.01). The results were similar in Jews and non-Jews. The combined analysis for the two blocks of IL17RA also displayed a significant trend for increased OR: H3 block 1/no H4 block 2 (OR 0.55), other, (OR 1), H4 no H3 (OR: 1.84, P Mantel-Hanzel <0.0001).

(1) IL17A appears to be an ethnic specific gene for CD; (2) IL17RA is a gene associated with both CD and UC. As is the case in mouse colitis, this cytokine/receptor pair could be important in the pathogenesis of a subtype of CD.

### Example 5

### An Interaction Between IL-23R and IL-17A and Between IL-23R and IL-17RA Haplotypes is Necessary for Susceptibility to Crohn's Disease

The inventors determined whether an interaction exists between IL-23R and IL-17A/IL-17RA for conferring susceptibility to CD development. SNPs were selected to tag common haplotypes and genotyped in 763 CD and 254 controls using Illumina technology. Haplotype blocks were constructed using Haploview 3.3. Analysis was done in the total sample first, and then in Jewish and non-Jewish subjects separately. Analysis for gene interaction was performed using the Breslow-Day test.

Two IL23R risk haplotypes were identified (IL23R block 3 H1 and block 2 H1) and one each for IL17A (IL17A H2) and IL17RA (IL17RA H4) to confer increased risk for CD. IL23R and IL17A interaction: while the risk haplotype for each gene contributed susceptibility individually, there was no increased risk for disease if either of the two genes' risk haplotypes were absent. IL-23R absent/IL-17A risk (OR 1.04, p=NS); IL-23R risk/IL-17A absent (OR 1.1, p=NS); however, the combination of the risk haplotypes from IL23R with the risk haplotype from IL17A dramatically increased risk for CD (30% in non-Jewish CD vs. 16% of controls, OR 2.4; p for interaction 0.047). IL23R and IL17RA interaction: IL23R absent/IL17RA risk (OR 1.1, p=NS); IL23R risk/IL17RA absent (OR 1.3, p=NS): i.e. no increased risk if a risk haplotype was absent. Yet again the combination dramatically increased risk in the total CD sample (OR 3.0, p for interaction 0.036). IL17A and IL17RA interaction: In contrast, the inventors found no interaction between the IL17A and the IL17RA haplotypes in non-Jewish CD (P=0.4). When all three haplotypes were examined sequentially for interaction, the OR for CD in the non-Jewish population increased from 1 when neither haplotype was present to 3.7 (CI 1.3-10.1, P_{Mantel-Hanzel}=0.0004).

The inventors' data demonstrate the multiple and likely complex interactions between the individual components of the IL-23/IL-17 axis, which therefore appear to be playing a significant role in CD mucosal inflammation.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

What is claimed is:

1. A method of diagnosing susceptibility to Crohn's Disease in an individual, comprising:
determining the presence or absence of at least one risk haplotype at the IL23R locus selected from the group consisting of IL23R Block 2 H1 and IL23R Block 3 H1,
wherein the presence of at least one risk haplotype at the IL23R locus is diagnostic of susceptibility to Crohn's Disease.

2. The method of claim 1, wherein the individual is a child.

3. The method of claim 1, wherein the individual is non-Jewish.

4. The method of claim 1, wherein the IL23R Block 2 H1 further comprises one or more variant alleles selected from the group consisting of SEQ. ID. NO.: 9 and SEQ. ID. NO.: 10.

5. The method of claim 1, wherein the IL23R Block 3 H1 further comprises one or more variant alleles selected from the group consisting of SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, and SEQ. ID. NO.: 18.

6. The method of claim 1, wherein the presence of two of said risk haplotypes at the IL23R locus presents a greater susceptibility than the presence of one or none of said risk haplotypes at the IL23R locus, and the presence of one of said risk haplotypes at the IL23R locus presents a greater susceptibility than the presence of none of said risk haplotypes at the IL23R locus but less than the presence of two risk haplotypes at the IL23R locus.

7. A method of diagnosing susceptibility to Crohn's Disease in an individual, comprising:
determining the presence or absence of one or more risk haplotypes at the IL23R locus; and
determining the presence or absence of one or more risk haplotypes at the IL17A locus,
wherein the presence of at least one risk haplotype at the IL23R locus and at least one risk haplotype at the IL17A locus is diagnostic of susceptibility of Crohn's Disease.

8. The method of claim 7, wherein one of said one or more risk haplotypes at the IL23R locus is IL23R Block 2 H1.

9. The method of claim 7, wherein one of said one or more risk haplotypes at the IL23R locus is IL23R Block 3 H1.

10. The method of claim 7, wherein one of said one or more risk haplotypes at the IL17A locus is IL17A H2.

11. The method of claim 10, wherein IL17A H2 further comprises one or more variant alleles selected from the group consisting of SEQ. ID. NO.: 19, SEQ. ID. NO.: 20, SEQ. ID. NO.: 21, SEQ. ID. NO.: 22, and SEQ. ID. NO.: 23.

12. A method of diagnosing susceptibility to Crohn's Disease in an individual, comprising:
determining the presence or absence of at least one risk haplotype at the IL23R locus; and
determining the presence or absence of at least one risk haplotype at the IL17RA locus,
wherein the presence of at least one risk haplotype at the IL23R locus and at least one risk haplotype at the IL17RA locus is diagnostic of susceptibility of Crohn's Disease.

13. The method of claim 12, wherein one of said one or more risk haplotypes at the IL23R locus is IL23R Block 2 H1.

14. The method of claim 12, wherein one of said one or more risk haplotypes at the IL23R locus is IL23R Block 3 H1.

15. The method of claim 12, wherein one of said one or more risk haplotypes at the IL17RA locus is IL17RA Block 2 H4.

16. The method of claim 15, wherein the IL17RA Block 2 H4 further comprises one or more variant alleles selected from the group consisting of SEQ. ID. NO.: 26, SEQ. ID. NO.: 27, SEQ. ID. NO.: 28, SEQ. ID. NO.: 29, SEQ. ID. NO.: 30, SEQ. ID. NO.: 31, and SEQ. ID. NO.: 32.

17. A method of determining a low probability relative to a healthy individual of developing Crohn's Disease in an individual, said method comprising:
determining the presence or absence of at least one protective haplotype at the IL23R locus selected from the group consisting of IL23R Block 3 H2 and IL23R Block 3 H6,
wherein the presence of one or more of said protective haplotypes at the IL23R locus is diagnostic of the low probability relative to the healthy individual of developing Crohn's Disease.

18. The method of claim 17, wherein IL23R Block 3 H2 further comprises one or more variant alleles selected from the group consisting of SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, and SEQ. ID. NO.: 18.

19. The method of claim 17, wherein IL23R Block 3 H6 further comprise one or more variant alleles selected from the group consisting of SEQ. ID. NO.: 11, SEQ. ID. NO.: 12, SEQ. ID. NO.: 13, SEQ. ID. NO.: 14, SEQ. ID. NO.: 15, SEQ. ID. NO.: 16, SEQ. ID. NO.: 17, and SEQ. ID. NO.: 18.

20. A method of diagnosing susceptibility to Crohn's Disease in an individual, comprising:
determining the presence or absence of one or more risk haplotypes at the IL17A locus in the individual,
wherein the presence of one or more of said risk haplotypes is diagnostic of susceptibility to Crohn's Disease.

21. The method of claim 20, wherein one of said one or more risk haplotypes at the IL17A locus is IL17A H2.

22. The method of claim 21, wherein the individual is non-Jewish.

23. The method of claim 21, wherein one of said one or more risk haplotypes at the IL17A locus is IL17A H4.

24. The method of claim 23, wherein the individual is Jewish.

25. A method of diagnosing susceptibility to inflammatory bowel disease in an individual, comprising:
determining the presence or absence of one or more risk haplotypes at the IL17RA locus in the individual,
wherein the presence of one or more of said risk haplotypes is diagnostic of susceptibility to inflammatory bowel disease.

26. The method of claim 25, wherein one of said one or more risk haplotypes at the IL17RA locus is IL17RA Block 2H4.

27. The method of claim 25, wherein said inflammatory bowel disease comprises Crohn's Disease.

28. The method of claim 25, wherein said inflammatory bowel disease comprises ulcerative colitis.

29. A method of determining a low probability relative to a healthy individual of developing inflammatory bowel disease in an individual, said method comprising:
determining the presence or absence of one or more protective haplotypes at the IL17RA locus in the individual,
wherein the presence of one or more of said protective haplotypes is diagnostic of the low probability relative to the healthy individual of developing inflammatory bowel disease.

30. The method of claim 29, wherein one of said one or more protective haplotypes at the IL17RA locus is IL17RA Block 1 H3.

31. The method of claim 29, wherein the inflammatory bowel disease comprises Crohn's Disease.

32. The method of claim 29, wherein the inflammatory bowel disease comprises ulcerative colitis.

33. A method of determining a low probability relative to a healthy individual of developing Crohn's Disease, comprising:
determining the presence or absence of a IL12B(p40) H1 haplotype,
wherein the presence of the IL12B(p40) H1 haplotype is diagnostic of a low probability relative to a healthy individual of developing Crohn's Disease.

34. The method of claim 33, wherein the IL12B(p40) H1 haplotype further comprise one or more variant alleles selected from the group consisting of SEQ. ID. NO.: 33, SEQ. ID. NO.: 34, SEQ. ID. NO.: 35, and SEQ. ID. NO.: 36.

35. A method of determining a low probability relative to a healthy individual of developing Crohn's Disease, comprising:
determining the presence or absence of a IL12B(p40) H3 haplotype; and
determining the presence or absence of Cbir1 antibody expression relative to an individual diagnosed with Crohn's Disease,
wherein the presence of IL12B(p40) H3 haplotype and the absence of Cbiri antibody expression relative to an individual diagnosed with Crohn's Disease is diagnostic of a low probability relative to a healthy individual of developing Crohn's Disease.

36. The method of claim 36, wherein the IL12B(p40) H3 haplotype further comprises one or more variant alleles selected from the group consisting of SEQ. ID. NO.: 33, SEQ. ID. NO.: 34, SEQ. ID. NO.: 35, and SEQ. ID. NO.: 36.

37. A method of treating Crohn's Disease, comprising:
determining the presence or absence in the individual of one or more risk haplotypes selected from the group consisting of IL23R Block 2 H1, IL23R Block 3 H1, IL17A H2, and IL17RA Block 2 H4, and administering a therapeutically effective amount of treatment to the individual if said one or more risk haplotypes is present.


SEQUENCE LISTING (36 sequences)

SEQ ID NO: 1 - DNA, 2826 bp, Homo sapiens
acaagggtggcagcctggctctgaagtggaattatgtgcttcaaacaggttgaaagagggaaacagtcttttcctgcttccagacatgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagttccccaggtcacatcaaaagcattccaacatgacacatggaattctgggctaacagttgcttccatctctacagggcaccttacttctgacaacagaggagacattggacttttattgggaatgatcgtctttgctgttatgttgtcaattctttctttgattgggatatttaacagatcattccgaactgggattaaaagaaggatcttattgttaataccaaagtggctttatgaagatattcctaatatgaaaaacagcaatgttgtgaaaatgctacaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctgggccatatgataagcatatgtttcagttctaccaatcttgtttccagagtagtgacatttctgtgctcctaccatcaccatgtaagaattcccgggagctccatgcctttttaattttagccattcttctgcctcatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtaggtggatcacctgaggtcaggagttcgagtccagcctggccaatatgctgaaaccctgtctctactaaaattacaaaaattagccggccatggtggcaggtgcttgtaatcccagctacttgggaggctgaggcaggagaatcacttgaaccaggaaggcagaggttgcactgagctgagattgtgccactgcactccagcctgggcaacaagagcaaaactctgtctggaaaaaaaaaaaaaa

SEQ ID NO: 2 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 3 - DNA, 1859 bp, Homo sapiens
gcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

SEQ ID NO: 4 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 5 - DNA, 3429 bp, Homo sapiens
ctgggcccgggctggaagccggaagcgagcaaagtggagccgactcgaactccaccgcggaaaagaaagcctcagaacgttcgttcgctgcgtccccagccggggccgagccctccgcgacgccagccgggccatgggggccgcacgcagcccgccgtccgctgtcccggggcccctgctggggctgctcctgctgctcctgggcgtgctggccccgggtggcgcctccctgcgactcctggaccaccgggcgctggtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaggcggtggcgttttaccttcagccactttgtggttgaccctgaccaggaatatgaggtgaccgttcaccacctgcccaagcccatccctgatggggacccaaaccaccagtccaagaatttccttgtgcctgactgtgagcacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaagggtgctgtcgccaccaagtgcagatccagcccttcttcagcagctgcctcaatgactgcctcagacactccgcgactgtttcctgcccagaaatgccagacactccagaaccaattccggactacatgcccctgtgggtgtactggttcatcacgggcatctccatcctgctggtgggctccgtcatcctgctcatcgtctgcatgacctggaggctagctgggcctggaagtgaaaaatacagtgatgacaccaaatacaccgatggcctgcctgcggctgacctgatccccccaccgctgaagcccaggaaggtctggatcatctactcagccgaccaccccctctacgtggacgtggtcctgaaattcgcccagttcctgctcaccgcctgcggcacggaagtggccctggacctgctggaagagcaggccatctcggaggcaggagtcatgacctgggtgggccgtcagaagcaggagatggtggagagcaactctaagatcatcgtcctgtgctcccgcggcacgcgcgccaagtggcaggcgctcctgggccggggggcgcctgtgcggctgcgctgcgaccacggaaagcccgtgggggacctgttcactgcagccatgaacatgatcctcccggacttcaagaggccagcctgcttcggcacctacgtagtctgctacttcagcgaggtcagctgtgacggcgacgtccccgacctgttcggcgcggcgccgcggtacccgctcatggacaggttcgaggaggtgtacttccgcatccaggacctggagatgttccagccgggccgcatgcaccgcgtaggggagctgtcgggggacaactacctgcggagcccgggcggcaggcagctccgcgccgccctggacaggttccgggactggcaggtccgctgtcccgactggttcgaatgtgagaacctctactcagcagatgaccaggatgccccgtccctggacgaagaggtgtttgaggagccactgctgcctccgggaaccggcatcgtgaagcgggcgcccctggtgcgcgagcctggctcccaggcctgcctggccatagacccgctggtcggggaggaaggaggagcagcagtggcaaagctggaacctcacctgcagccccggggtcagccagcgccgcagcccctccacaccctggtgctcgccgcagaggagggggccctggtggccgcggtggagcctgggcccctggctgacggtgccgcagtccggctggcactggcgggggagggcgaggcctgcccgctgctgggcagcccgggcgctgggcgaaatagcgtcctcttcctccccgtggaccccgaggactcgccccttggcagcagcacccccatggcgtctcctgacctccttccagaggacgtgagggagcacctcgaaggcttgatgctctcgctcttcgagcagagtctgagctgccaggcccaggggggctgcagtagacccgccatggtcctcacagacccacacacgccctacgaggaggagcagcggcagtcagtgcagtctgaccagggctacatctccaggagctccccgcagccccccgagggactcacggaaatggaggaagaggaggaagaggagcaggacccagggaagccggccctgccactctctcccgaggacctggagagcctgaggagcctccagcggcagctgcttttccgccagctgcagaagaactcgggctgggacacgatggggtcagagtcagaggggcccagtgcatgagggcggctccccagggaccgcccagatcccagctttgagagaggagtgtgtgtgcacgtattcatctgtgtgtacatgtctgcatgtgtatatgttcgtgtgtgaaatgtaggctttaaaatgtaaatgtctggattttaatcccaggcatccctcctaacttttctttgtgcagcggtctggttatcgtctatccccaggggaatccacacagcccgctcccaggagctaatggtagagcgtccttgaggctccattattcgttcattcagcatttattgtgcacctactatgtggcgggcatttgggataccaagataaattgcatgcggcatggccccagccatgaaggaacttaaccgctagtgccgaggacacgttaaacgaacaggatgggccgggcacggtggctcacgcctgtaatcccagcacactgggaggccgaggcaggtggatcactctgaggtcaggagtttgagccagcctggccaacatggtgaaaccccatctccactaaaaatagaaaaattagccgggcatggtgacacatgcctgtagtcctagctacttgggaggctgaggcaggagaattgcttgaatctgggaggcagaggttgcagtgagccgagattgtgccattgcactgcagcctggatgacagagcgagactctatctcaaaaaaaaaaaaaaaaaa

SEQ ID NO: 6 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 7 - DNA, 2347 bp, Homo sapiens
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatc

SEQ ID NO: 8 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - DNA, 692 bp, Homo sapiens
ctggggctctgaaattgcttaggaccattttaagcaccctcaaggccataaatttctcacctcctcctgtcacccacctccacctctgagttcggcttggccactgttatagcagcacaagcattctaggacccttttggcaaaagaattattctgaggagaaagtaaaaatctgtttagtcttatgagaaatgcagatagcayagtaagaatcacagcataaagcaggtcagtgcaatccagatttaagtctttaagtttgaatgagttcatatttttgcaaactggcatttattatgtaatacatacttgaatatttagtttgttacacaagactcagatgttgaatttttattcttactgattaggtttcacatatttccaccagatcttacattttaaaaaagtatcgggaggtcgaggcgggccgatcatgaggtcaggagatcgagaccatcctggctaacatggtgaaaccccatctctactaaaaatacaaaaaattagcggggcgaggtggctggcgcctgtagtcccagctactcgggaggctgaggcaggagaatggcgtgaacccggaaggcggagcttgcagtgagccgagatcgcaccactgcactccagcctgggcgacagagcgagactccgtctcaaaaaaagaaaaaaaaaagtatcaattttattatagatgt

SEQ ID NO: 10 - DNA, 705 bp, Homo sapiens
ttgtttgcatacacttaaatgggatccacgttctgcatcatttgattgataatcaagtgaagatcctgctgaattccttttgcatatgcagaatttagattaaatttcaaaacaacacaaatacaattctcaagtcytagattctgaattaatggggttttatcctaataagacacctggggtccttgtatagtatcacagtcatagaatgatattaaagaatactgagtttcttaggctgggtgcagtggctcatgcctgtaatcccagcactttgggaggccaaggcaggcggatcacctgagctcagggattgaagaccagactggccatcatggcaaaaccccgtctctactgaaaatacaaaaaatttagccaagcctggtggtgtgtgcctgtaatcccagctactcagaaggctgaggcaagagaatcgcttgaatctgggaggtggaggttgcaatgagccaagatggagccactgcactccagcctgggtgacagagtgactctgtctccagaggaaaaaaaaaaaaaggataccaaatcctcttacttcatgcaaataggagtatgtaatagactagaaaaagtgtttagaaaatagaaaggaattatattatcagtgtctctgaataagttttcagaagccaactgttttctggttgaaactcttattctctgctccccctggtggtgctacat

SEQ ID NO: 11 - DNA, 1077 bp, Homo sapiens
cagctaccatttctccaccccattaaaagagtatattccaaaattaagaatatattccaaaattaagaatatattccaaaattaaggctgggtatggtggctcactcctgtaatctcaacactttgggaggccaaggcagagagatgacttgtgcccaggagaccagcctgggcaatataatgagaacttatctctacagaaaaatttaaaaattatccaatcatggtagtgcatgcctgtagtcccagctacttgggaggctgaggcaggaggatcacttcagcccaggaggaggtggaggttgcagtgagctgtgatcgagccactgcactccacagtccagcctgggcaacagagtgggaccctatctagaaaaaaaataaaataaaaaatatatatatacacacacacacatataaataaataaatatatatacacacataaataaatatatatacacatatatataatatcacatttggactttctggagatttgagacagttgtcaaacataaagcagtatgggctgggcacggtggctcacacctgtaatcccagcactttgggaggccaaggtgggcggatcacttgaggtcaaaaattcaagaccagcctggccaacatgatgataccccatctttactaaaaatacaaaaaagtagccaggtgttgtagtgcatgactgtaatcccagttacttgggaggctgaggcagaagaatcgcttgaacccgggaggcggaggttgcagtgaactgagatcgagccaccgcactccagcctgggcaatagagcgagactccatctcaaaaaaagcagtgtgtgtttcagttttaatgtatttcagagacagtatttgattatgtacggccaygttttatataaagaacactttgttttcctagagtctagaagacagcttggaacataataggtgttccatacatttctgctaaataaaatagttgttttaaaagcacaccacattttattattgttacccatccattttaggttaaagaatttgacaccaattttacatatgtgcaacagtcagaattctacttggagcca

SEQ ID NO: 12 - DNA, 997 bp, Homo sapiens
catttctgctaaataaaatagttgttttaaaagcacaccacattttattattgttacccatccattttaggttaaagaatttgacaccaattttacatatgtgcaacagtcagaattctacttggagccaaacattaagtacgtatttcaagtgagatgtcaagaaacaggcaaaaggtactggcagccttggagttcacygttttttcataaaacacctgaaacaggtgagtgtacttatatattttattctgttgggcttttctttatatatcttttctgctgagcacagtggctcacacctataattccagcactttgagaggccaaggcaggaagattgcttgagcctaggagtttgagactggcctgggcaacatagtgagaccctagtctgtacagaaaaataataattattattagcctgggtggtagaatgcatttgtagtcgcagctacttgggaagctgaggtagtaggattgcgtgagcccgggagtttgatgctgcagtgagctatgatcatcccactgctctctagcctggaggaaagaccaagaccctgtttcctaaaaagtttaaaacagccaggtgcagtggcttatgtctgtaatcccagcactttgggaggccaaggtgggtggattaccttaggtcaggacttcaagacctcctcggccgacatggtgaaaccctgtctctactaaaaatacgaaaattagctgggcatggtggcaggtgcctgtaatctcagctactcggaaggctgaggcaggaaaattgcttgaacccaagaagtggaggttgcagtgaactgagattgtaccaccgcactccagcctggccaagagagagagacttggtctcaaaaaaaaataaaaataaaaataataataataaataagttaaaaacaaaataaagctacaagatattttttttctctttacctttgaccaaaattgacaaaactattctagggcagatgataacatttaaat

SEQ ID NO: 13 - DNA, 720 bp, Homo sapiens
ccagtgtgaaaatactgtgcattttccccaccatccctcagcaatttcattctttaatttcagggaagcagaggagcaacttacttaagtattctaagtataggactacaaatgttcttctttaaacataaaagtcttggcgaggtgtggtggctcatgcctgtaaccccagcactttaagaggccaaggcgagtggatcacctaaggtcaggagtttaagaccaccctggccaacatggtgaaaccccgtctctactaaaaatacaaaaattaactgggtgtggtggcaggtgcctgtaatcccagctactagggaggctgaggcaggagaatctcttgaacttgagaggcggaggttgcagtgagccaagatcctgccactgcactccagcctgggtgacagagcgagactctgtctctaaataaataaataaataaagtaaaataaagataaaagtcttaagcttcaggtagaaggaaataggaacaccacagtttaaatttaaggtctgtttcctraggagaaaaatcacttaagagacaaaaataccaattaaaattaagtatccctgaaaacttggatttattaaagtttaacatgttagctaagagaaaccatagactgttctcttggtacaaattcccttctaagacacattacatgagaaacagtaaaagtgtgttagggaaagtgctcatgttaaatctctttgaaaatg

SEQ ID NO: 14 - DNA, 1001 bp, Homo sapiens
cccatacacatgttggtaatcagaggtcacagaagtgacctgtgttgtgaaagtactatatagcaagagaaattgagtatgttctttctactcagttaccttataaggcaaaagggaattgagaggaagtggctatcctagattacatgggtggatctggtaaaatcacaagagttcttataagcagaagggagaaggttgagagtcagagaaagagattggaagatgctatgcttctggctttgaaaatgaaggatggagccatgagctgaggaatgtaggcagcctctagaatatagaaaaagcaatgaaactgattctgtcctgtagcctccagaaggaacataaccctattgacaccctgatttcagcccagtggttgtgattttggatttctcacctccagaactataagataataaattcatgttgttttaagctttcaagtttgtgatgatttgtgacagtagtaataggaaactaatatagaagatgatgacytcaagaaaaagcataatcataggccaggcatggtggctcctgcctgtaagcccagcactttgggaggccaaggtgggcagagttcttgagtccaggagttcaagaccaacttggcaaacatggtgaaaccctgtctctacaaaaaaaaaaaaaaaggaagaaaaaaaattagctgggtatggtggtgcatgcctgtagttccaggtacttgaaaggccaaggtgagaggattgtttgagcccagatcttatgagctgagatcacaccactgcactccagcctgggtgacagagagagaccctgtctaaaaaagaagggaggaaggaaggaaggaaggaaggaaggaaggaaggaaggaaggaaggaaaaagaaagacagaaagaaagaaggaaagaaagaaagaaagagagagaaagaaagaaagaaagaaagaaaagaaagaaagagagagacagagaaagaaagaaagaaagagaaagaaagaaaagaaagaaaggaa

SEQ ID NO: 15 - DNA, 701 bp, Homo sapiens
aggtgcggtgcctcacacctgtaatcccagcattttgggaggctgaagcaggtggatcacctgaggtcaggagttcgagaccagtctgaccaatatggtgaaatcctgtctctactaaaaattccaaaaaaaaaaaaaaaaaaaaaaaagccacgcgtggtggcatgctcctgtaatcccagctacttgggaggttgagacaggagaattgctagaacccaggaggcagaagttgcagtgagccaggatcatgccactgcactccagcctgggcaacagagggagattctgtcttaaaaaaaaaaatccggttttgattatgtcttcatagcagtgtgaaaacagactagtacggttgatgtagaaagaagagctgaggtgatgatttggcatcatccttaaaatacagatggaatacgttattgctaaaaccaggtccttttgagtggatttgattaaactagcctggtgttttggtaggccaaaaaatatagttgttaygctttaaattttgtccaacaataagaaaccatatttctcgtttgagatcactctaaattcccacaggcacattgtcttcttgtaagactaaagtttggtgccagtgtgtacaagttatataaaaattcttcccaaattaaagataatttggattttttttagtatattcaagtatgtcctgtgagattaataggcataag

SEQ ID NO: 16 - DNA, 886 bp, Homo sapiens
tatttgaagcaactaattgggggtactggctgccacacacccttgggcattaattagtgcctggaagaggatagacagccctcaggtcaacacagtgctcggcaaaggggtctaagcagtagagcagaatgaccaagagcgtggcctgatatacctgggtttgaattaaactctgcctcttatcagctctgtgaccttggggcataattatgaacttgctgagtctcaggttttctcttttggaaaatagagataataatacttatctaacagagctgccatgagttcctaacctccactgatcccacagaaatatcaaggtgtaggtaggtctgtgtaggcatctataattagggaactgtactgaacctaagcacttggcttgyaattgattgataattcagagtgcccttacctttcttcatgtttctttttctttttcttctttttcctcttttttttttttttcctgagacagggtcttgctctgttgcccaggcgggaatgcagtggagctcactgcagcctctatctctggtgctcagttgatcctcccacctcagcctcccaagtagctgggactacaggtacatgacaccacacccatctaatttttgtattttttgtaaaaatggggttttgccatgttgtgcaggctggtctcaaactcctggactcaagcaatctgcctgccttggcctcccaaagtgctgggattacaaaatgtgagccaccatgcctacccacttcatgtttctttacgacacttcaccaccacctgacttttcttcttgttttgtttgctgtttttctgccctgtctggctagaatagaagctccatgaagacaggggctttgctcattgttttcactgctgattccccag

SEQ ID NO: 17 - DNA, 601 bp, Homo sapiens
ttctcaaacaaaaagttgtttcctggggtagttgtgcactctggaaaaacagtcactctgtggcctaaagtaaaggttaattttgcttccccccaccctttctcctttgagacctttgctttgagcagagtaaagagaatagtaattctggtatcaaatgaagactaatgcttggttaaaattatttttctttcctttcattagacaacagaggagacattggacttttattgggaatgatcgtctttgctgttatgttgtcaattctttctttgattgggatatttaacagatcattccraactgggtaggtttttgcagaatttctgttttctgatttagactacatgtatatgtatcaccaaaatttagtcatttcagttgtttactagaaaaatctgttaacatttttattcagataaaggaaaataaaaagaacaatgtttaataagtacttacccatgccaaactctctacaaatgtctttcctttaatcctcaaaatgaccctgccagaaaagcttcctggcctattttacaggtgacttaaatgaggcttaaagaggctaagtcctcagcccagaatcactgaacagtaagcc

SEQ ID NO: 18 - DNA, 601 bp, Homo sapiens
tttgaacattaaaatatttcaagggacttcataatcaagtatattttaaaacagcctcaaataaaattccgtattagtttgccttccttacaagggtattaggaatatgtttattaatgtgtaatttaaattttgaaatattaagttctgagcaaaaaacctatgtagataagaaatcattagtagactttataatagctcatttaaaatctttctactgcacttgattataaatgtaaacgaaagaaagattatttcatgaagcaaatgatggcaagaaggagaaactcagtgccaattyggcaaagaacattcaagtcaaaatttgtgagcaactggacacactggggaactgccacaccaaacaactctaatctatcgagcagcttagaaatactcaatgcatcagtaaaatttagaaatccaagggtcttgcttttctcaaagtctcattttaaataactaaccatagatctttactaataccatcacaggagggaaaaaactgaagggggccaagagtaagggactttggggctgaatgctaaaacactaaaacaattggtaaggaattgacaaatttaaaaattgtcacacttcc

SEQ ID NO: 19 - DNA, 601 bp, Homo sapiens
ttgagtagtttccggaattgtctccacaacacctggccaaggaatctgtgaggaaaagaaagatcaaatggaaaatcaaggtacatgacaccagaagacctacatgttacttcaaactttttcttcctcatgaaccattaaaatagagcataactcttctggcagctgtacatatgttcataaatacatgatattgacccatagcatagcagctctgctcagcttctaacaagtaagaatgaaaagaggacatggtctttaggaacatgaatttctgcccttcccattttccttcagaagragagattcttctatgacctcattgggggcggaaattttaaccaaaatggtgtcacccctgaacccactgcgacacgccacgtaagtgaccacagaaggagaaaagccctataaaaagagagacgatagcgctacattttgtccatctcatagcaggcacaaactcatccatccccagttgattggaagaaacaacgatgactcctgggaagacctcattggtggtgagtcctgcactaacgtgcgatgctcttgctgatttggaccagatagtatttctggaccgtgggcatgaaacgct

SEQ ID NO: 20 - DNA, 801 bp, Homo sapiens
aatagagcataactcttctggcagctgtacatatgttcataaatacatgatattgacccatagcatagcagctctgctcagcttctaacaagtaagaatgaaaagaggacatggtctttaggaacatgaatttctgcccttcccattttccttcagaaggagagattcttctatgacctcattgggggcggaaattttaaccaaaatggtgtcacccctgaacccactgcgacacgccacgtaagtgaccacagaaggagaaaagccctataaaaagagagacgatagcgctacattttgtccatctcatagcaggcacaaactcatccatccccagttgattggaagaaacaacgatgactcctgggaagacctcattggtggtgagtcctgcactaacrtgcgatgctcttgctgatttggaccagatagtatttctggaccgtgggcatgaaacgctgggttctgactatggagatccaggaatactgtatatgtaggataggaaatgaaagctttggtaggtatttaagtcattgtgcagcattttcaagaactgatacacagcagtttgaaagataagattaaaactgaaagatagctatattggggctaaaccacacaagaagtgtcacatgatgctgtgcagtaagaaagaaaatttattgaaagtctgtttttctgagtacaaaggatttaatataattctcccacggcatttttctttaaaatgggtcactatccttgagattttgaaagccgtagcagcaacaacctttgtttccattatctcgtaccata

SEQ ID NO: 21 - DNA, 511 bp, Homo sapiens
ccatggctttaaaatttttttaaaaaaactagtttcaacattctccttttgacttaggaaagacatgttatccattggttggcaataattttaataaaaatgtcaagtcatggcatgtcattagcctatcagcacatgcatcattgtcaggtctgggaaggaataataaccttgattttctaggtagaaatatcctcctgcaccattgttctcagtcccatattctgtgaaactcatcgtgaagtcaaacattcamattggaagaaagagctatagaaaatctatgtggtatcaatattcatgctagaagtgctgttggtgctactggcaggcatccaactaaaaactcgatctccttcatgttttcttaggtatattttccagttgttctaaatttaacatgtattgattctgtaataaaatcagatttcaaaaaagatacttgaagttaaatatttaaaaaatataaaccccacttattctaaaacacagttatacctatgtttagtta

SEQ ID NO: 22 - DNA, 511 bp, Homo sapiens
ggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttrgccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagc

SEQ ID NO: 23 - DNA, 1293 bp, Homo sapiens
gaacctgggtagtatggtattggtggggaggtgggggttccttggagaacttttggaagtgagaatatagtatttggtgatatgtggatgttaggaatgagggagaggcagaaggaaaaagattcagggaagccacatagatttctagcttggatgactaggtacatggtagtgctaactggggaaaatgaagagagaataaaagcaaagtgtatcaggggaggagtacatggaaagcaactgcctcttcccatccgcataccccccacccaaaatctagtgggaaataatggttcaggaccacacacacacacacacacacacatatagacatatacatcctttacaactccctctcccaacaaaaacaaaaacaattttttcttttcatcatcaccgttcagagaaagcttgaaaacgagcagcaggtttttagtgagaagcttgaaagcgtaaaggctgtgaggaactgtccctggaagctgcctggggatttcctgtaggaaaatggtgacagggatggtcacaggaatcaagatgtgagcacaaaatgactgagaggaggtggctggagaggccaacccctggatttggaatagggaaagaagcctagaaaagccatgggcctctgggtgggctggagcacactggatggagcaggatggagtgaagaggaaggtctttcaagaagcagggagcctgcagagtggcctgagaatatctagaggccttcagaagtagggcaagacagcacatgggccatgggggcgaaaatggttacgatgtgaaacttgaaactactctggaattgaatgtgattgagtttttattttacttgggctgaacttttctcatacttaaagttcrttctgccccatcagctcctttctgggttgtgtggtgccttgatcagacagaagccaggccctaggagtgttgcttgaggaagagaaaaatgttggtctgttgatctctgaggggccttaatctccaaaggaagcctgagtctaggggagaaactggacattgtagtctgaagacaatgtctcctcccagaacttcttgtattttggggagggtttcattttccccatatgatctttaataatgacatgccattcctcagggccattatcttatttgctctattcctatcaaaatgcttctgtctacagcattggctaatagtatgaaaaccttagtcggtgttcagtcttgaaggcatgtgaaatcgagaacttggaattttgggtatttccaggtcattgttactcaaagacatgctttgttttg

SEQ ID NO: 24 - DNA, 401 bp, Homo sapiens
aatctccaggccaccagaaactgctgctttcagccctctcagagcacagccaaacttccccctcagtcccagtgggggactcagtctccagtaagtacatccttcctgctacctatgtctcagtttcccaaattctagaaagcacagagaattgctcacaaaggaatccaaagccaaggcctgacgggcttttatcttaamggaacatgcgtatgagccttctggtgacagcaattagagcagccaccttgaagcaatgtgacacagtcccacctttggccgctgagtgattgcagacactcattttgcttgtctgtggtggagagaggtctctggcctcctgctttgaggctgcagccacagcttgcctggccctgtgtaagtgtttgacctatttcata

SEQ ID NO: 25 - DNA, 905 bp, Homo sapiens
tgtatatggtcaatgcgctattacctctaatagaattgttattctatatttcacctatatgtatatttttcagttttgattaaatcttttttccccttatcttccctcatgccatctcctctgcttgtgtgccctttgcccccagcaaactgtgttaacacctgttaacatgtttctattctcatttagtcatacacacactctctctctctctctctctctctctctctctctctatatatatatatatatatatatatattcagggagatctttttcatgacttatttccatagaaattgggatcatactataaaaagttatttgaaacttattttcctctctcatcaacacattccagccatctgcaggtcaacagatgtctatgcaactaattctcattctctttaatatttgcataatattccatagtagatagcaatctattcaaccrtttctagtttgatggacatttagatttaaactagatctcaccttaattcagccattctctattgatgaccattcagtaccacagaaaaagagggaaagctgtccattttttttaaagctagtataagcttaattctaaaacttgacaaatcttatacaaaaagaaaaactatggaccaatctcatttatgaacataaatccatgcaattctaaataaaatattagcaaatgtaatctagcagaatatcaaaagaacaatgcaccataatctagtggtgcattaacaaactttgtttgttaatcagtgctggagaggtatatgatgaggttcagagctgttttgaatgaaaactataaacctaatagtaactgaaggaaaagacttatatgcaaccacaactatcagaaaccaagaccaaaaacagtattccatagggcataccaaactatt

SEQ ID NO: 26 - DNA, 601 bp, Homo sapiens
tggggagcgtgcacaggtggagagtgtggtgtggctggagtggggagcgtgcacaggtggagagagtggtgtggctgggagtggggagcgtgcacaggtggagagagtggtgtggctgggagtggggagcgtgcacaggtggagagtgtggtgtggacgggagtggggagcgtgcacagtctggcattcttgctggtggacaggggaaagcttgtcctctctgtggcaccaagcaccactaccagtcaggattccttgcctggtaaggcactgcccctgcctttctcctgtctggttctcycaccctcacctgggcaggggttcgctgacccgcccttgctggagggagatgatggtcacctggagatcgtggtgtagccagccaggatcccctcctctcacattgccgctgctggctggaaggcatgggcgctctacagttctggagcccttttcctgccctctctgcccgcagatccagcccttcttcagcagctgcctcaatgactgcctcagacactccgcgactgtttcctgcccagaaatgccagacactccaggtaggggacatgcggctgtcctaggccatactgggagaaca

SEQ ID NO: 27 - DNA, 801 bp, Homo sapiens
gcactgcccctgcctttctcctgtctggttctcccaccctcacctgggcaggggttcgctgacccgcccttgctggagggagatgatggtcacctggagatcgtggtgtagccagccaggatcccctcctctcacattgccgctgctggctggaaggcatgggcgctctacagttctggagcccttttcctgccctctctgcccgcagatccagcccttcttcagcagctgcctcaatgactgcctcagacactccgcgactgtttcctgcccagaaatgccagacactccaggtaggggacatgcggctgtcctaggccatactgggagaacaagtggctgaaggcccccagcctgtgctgcgtccttacctggttctgaggggtgattagggaggagastttagtttaacttggagtccttcaggcctgaagtgtggagtggggctttagagtgtcactccctggggctggactcctggctgtctttcattagctatgtagccttaggcaaattacttaatctttttgattctcaacttccttgactggaaaatgaggtggtttttatcctagagccctagttctgtgccatgcactgagcgcagtgctccaacatgccgtccattttttcatcctcactcattgtgagtcacggtactatgcagtagaggatccccccaccccaaaccccaggttcctggataaggaaactgaggcacagagatgttgaataacttgtccaagatcacacagcagggacgctgttttcaaaagtcgcatgccctaatgcacgggaggc

SEQ ID NO: 28 - DNA, 801 bp, Homo sapiens
gcagtagaggatccccccaccccaaaccccaggttcctggataaggaaactgaggcacagagatgttgaataacttgtccaagatcacacagcagggacgctgttttcaaaagtcgcatgccctaatgcacgggaggctgcagccacgtgctcaccagaaggcaaggcgcaggcatggagccaggctggaaggagaacccagcctcccaaggaggaggcaaggtgtctcttcttagaccagcaactcaagtgtctcttgtagatggtttcattaagttcaacctggatctagagtgcctggtgcagggccaacatcattaaagccctcaagggacgtcagttgtgtttcttgtgatgactgggaagggttaagaatgctattttccctttttcctctgttytcattgcagaaccaattccgggtaagcttggatctctctccgacagcactgcagccctcaggggacattccccagtggccacttgagaagtccctgcctcagccaggcagacaaggctgaaccgaggccagcccggggtggggggtgagaccatggtttgtcgtggtggggccagagaggacagagcctggggctggggagcagggctgggggcctcagggtgggcagggcaggccccgccgcatcactcacgctgttctgctcaccgcagactacatgcccctgtgggtgtactggttcatcacgggcatctccatcctgctggtgggctccgtcatcctgctcatcgtctgcatgacctggaggctagctggtaagcgctggggctctggctgtcctg

SEQ ID NO: 29 - DNA, 801 bp, Homo sapiens
ccagccccacctcattagccttgtagtcacaggccagttacttaatacaccatggattcacttttctgtaaaatgtactgataatgcctccctctaagggtgtgacgaaggttaaatgagtagctgaggaaggtgcttgctggtggggattagtacataccagtgtcttctcccacctgcagccctctgctggccaagtcctaagccgggagaacacaggccttccggttggggcttcagcccttgcctgccccaccatgaccctaggctgctccttccgtcatctgggaagctgtttccacccttccctaggctcgtcaggattaggtgttaatcattattaattattatgtggtagaaagaaaaccagccaggcatgggaggacctatgggaggttccrataacattcagtagcatctcggccagtgctccacaggcggtgcagctctctaaaggtttggggctgggcggcggcggcgcttttggtttcctttctgctgttgcgcttctgttttccgaagtgtcctgcaccacagggtgaaggcaagaggagcctcgctgttatttggctgtcttgtgacagttctggggaagagctgaaagggttaggattgagattaaggttctaagtcgtttgctcagtcatctgtggatctcaatcctcccagctgtcactaaggagttaacccccgcagagcagttttttcatcacatctctgaggggaacaattgcttaagtatgtgggttccccttcctcacctcaaaaataccaggaggaaatgttgcaagcagcctggtg

SEQ ID NO: 30 - DNA, 768 bp, Homo sapiens
tcagtgctacaaaataactgtgatcccaattgatwatgtacaacgtgccaggcacgtcacatacacacactcatttaatacccattaaacaagagcaaatacagacccacctcacagaggaagaagctgcatttcagaggcactagtaactgctccaggtcatagtgctcgtagtggcagacccaggactcatgcctgtgcgaccacctagcacggcctcgctgctcagtctcggggctgcccccttacccttcaccctttgtcagggatggggcagacaccctgtgagctggtttctatttctcttcccaaagaaccactccagtgtatttcttttcctttccagggcctggaagtgaaaaatacagtgatgacaccaaatacaccggtcagtatttcctggtttgcatgtttgcttatttttaaagcagtggagggttctcctgggataagtgcgtgggtcgcctcctgtgctctaactcccaagtcccttcaggagaccccaccttagaaacccccttccagtaccccactcagaagggcccccaataacaaggcctggtgccattttttgaatcactcagacaaggaaagaaaggtaagtattttgtgaacagaggtcctccctggagcagaccaacagagcttggtgcctctttttcttttcttattaaagataggctaagacagctgggacgttgagagatcttttccacaggcagcagactgattttttcaggcagcaagggtggatagtacatctgggtt

SEQ ID NO: 31 - DNA, 829 bp, Homo sapiens
acctcgctgaagtagcagactacgtaggtgccgaagcaggctggcctcttgaagtccgggaggatcatgttcatggctgcagtgaacaggtcccccacgggctttccgtggtcgcagcgcagccgcacaggcgccccccggcccaggagcgcctgccacttggcgcgcgtgccgcgggagcacaggacgatgatcttagagttgctctccaccatctcctgcttctgacggcccacccaggtcatgactcctgcctccgagatggcctgctcttccagcaggtccagggccacttccgtgccgcaggcggtgagcaggaactgggcgaatttcaggaccacgtccacgtagagggggtggtcggctgagtagatgatccagactttcctgggcttcagcggtggggggatcaggtcagccrcaggcaggccatctaaggaaacaagaccacacatgctgaccctcaccccagggcccagggcagctctgtgcctgccagcccaggaggggcctggaccaggacacagagcttggctccctccctaagctgagaaacccaactgaggcctgttggaaaaacccagatgttactattccacctttggctgcctgaaaaaaatcagtctgctgccttgtgggaaaaagaatctctcaacgttcctagctggtctttagcctatactttaattaagaaaagaaaaagagcaccaagctctgttggtctgctccagggaggacctctgttcacaaatacttacctttctttccttgtctgagtgattcaaaaatggcacccagcttgttattggggcccttctgagtgggtact

SEQ ID NO: 32 - DNA, 852 bp, Homo sapiens
tgtttagccctcagcctctctccatgcagaggctcatcagacgaaaggtgccccaggcctcaggactgatgcgcacaaggctgtccccacccctgagctctggcgacatccccccaacccccaccccgatctctctcactgcctccctccttcccctccaggctccaccagcagctccctgacaagctcactccactcacctcccagcacttacccacaaactgcttccttgctgggactacgctttccccaaccacaatccttcasctcaggcatctcctcggggatcccccctgacctgggtgcctttcccgtgcatgctcacaaccctgggcaggcttccactccatctttctacttttttattttttttgagacagggtctcactctgctgcccaggctggattgcaatggtaccattatagctcactgcagcctctacctcctgggctcaagtgatcatctggcatcagcctcccgagtagctgggactacaggcatgtgccaccatgactgactaaaaaaaaatttaggtagagatgaggtctcactatgtcgcccaggctggtcttgaactcctgagctcaagcaatccacctgcctcgccttcccaaagtgctgggattacaagcatgagccactgcacctggcccattcagcgtttacatcccgcgtgaccatcttttttttttttttttttgagaagagtctcgctctgtcatccaggctgcagtgcaatggcacaatctcggctcactgcaacctctgcctcccagatcaacattctcctgcctcagcctcccagtagctaggactcagcatgtgttaccatgccccggctattttctatt

SEQ ID NO: 33 - DNA, 601 bp, Homo sapiens
tccaggccacataaggaaggcctgggccttctggcatgaaatccctgaaacccagttgcccaggatcatatgttgtgagaaataagaagagacattgctgttacaatgtcaccccacatcaacttttggcattctcttccaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttmgatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttct

SEQ ID NO: 34 - DNA, 601 bp, Homo sapiens
ggcaaggcaattgtgctagaaagatgaaagctgggccaaacgatttctccctcaagggcttacaaagtacaaaagctgcacctacatgtggagtgtctgccagtaggtggtgcaagttctatgcacacccctgtgaattgcaagcacagtgccctaagaccaagatgggcttgttttgggagagtatgcattgcagaaacaggctcagcttaccctgtgactatgttgccaaggggtcttcacagctttccttctcttttgcagaaagatagagtcttcacggacaagacctcagccacgktcatctgccgcaaaaatgccagcattagcgtgcgggcccaggaccgctactatagctcatcttggagcgaatgggcatctgtgccctgcagttaggtgagcaggccctcaaaggccagcccaggcctgcactctcagtgcacctggatgcagggatatgattgggggctgtgttggagaggaaagggggatggagtggccagcacccagttgccagaatcagaaacatacatttattcactaacagatatttatttggtgcctttgttatgtaggacactgtgctggccacagggatatt

SEQ ID NO: 35 - DNA, 601 bp, Homo sapiens
ctctggtttctcagcatttttctagaactatttcattaagaaattaagggcaacctctcagtgacctatcagttaatgataatgggaaaagcaaagtcaaacccgtgttttttcaaccgcccttccttgtctacattgaagaaagaacatggagattttagccgattgcttgaataaatgtatgtgttggggcaggatattattgggaactgagaatagtctctgctgtgtttgaacccactcatccaaattgcctggccatgcttcctgaagcctcatagcaccaaagaaagggataaamggagaattcaaagctacaaatgacttgctgaaattgcaccttgagtcaaaaataaaaacaagagctccagggcgtagatcttaggggccctgaagcagactccaaaactcgatgaggcctcccgaaattttcccagggccacctcaactccttttacttctgctgacaccactaatctgaagttcgctgttggtccaatgcacctggactttccgtaagaaagcaacttccataaatacaagacctatgtgttaacccccatgtggcttactttaatcatcaccgaagcaaaccccaggt

SEQ ID NO: 36 - DNA, 801 bp, Homo sapiens
ctgctgaatattgtgccctgccgtattctctatgaaactgaaattgtgctggaagtttctctcccccagacctttggcaaagagtcttgtgctgtttgcagtttttggtatattaaggtgtttccaatctgctaaataatcaaaggttactattaaaggcagccttccagtcaatgagtcgatggcagctataaaactctttgtttctcttttccatgaccttgagcccaagcagggtctcatgccttgagatcatctcagcaagcatttgccaaatacttgttgtaaacaaggttgtgtttaggcaatggggatgcccgaagggttaataaaacacagtcccagagttcctggagcttacagcctggttctccactttatgtgcattccagtttatgtcrtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaaggtaattctatacccttggatagtatcaattttctctttcgctcataagagttaaaaacaacaacaacaacaaattgaaaagccaagtcatggtgagtgtaatgaattaacatcaagtctcttattgatgttaa